SEQUENCE LISTING (33 sequences)

SEQ ID NO: 1 - protein, 8 aa, Artificial sequence
  Synthetic construct
EHALYDAL

SEQ ID NO: 2 - protein, 8 aa, Artificial sequence
  Synthetic construct
YAFNFELI

SEQ ID NO: 3 - protein, 9 aa, Artificial sequence
  Synthetic construct
ITILQSWIF

SEQ ID NO: 4 - protein, 22 aa, Artificial sequence
  Synthetic construct
PKKKIQLHAEHALYDALMILNI

SEQ ID NO: 5 - protein, 22 aa, Artificial sequence
  Synthetic construct
LEDYAFNFELILEEIARLFESG

SEQ ID NO: 6 - protein, 21 aa, Artificial sequence
  Synthetic construct
EDEQEEMANAIITILQSWIFS

SEQ ID NO: 7 - protein, 19 aa, Artificial sequence
  Synthetic construct
KDEAEKAKRMKEWMKRIKT

SEQ ID NO: 8 - protein, 100 aa, Artificial sequence
  Synthetic construct
PKKKIQLHAEHALYDALMILNIVKTNSPPAEEKLEDYAFNFELILEEIARLFESGDQKDEAEKAKRMKEWMKRIKTTASEDEQEEMANAIITILQSWIFS

SEQ ID NO: 9 - protein, 100 aa, Artificial sequence
  Synthetic construct
  MISC_FEATURE (23)..(33): Xaa can be any amino acid
  MISC_FEATURE (23)..(33): Amino acids 23-33 may be present or absent
  MISC_FEATURE (56)..(57): Xaa can be any amino acid
  MISC_FEATURE (56)..(57): Amino acids 56-57 may be present or absent
  MISC_FEATURE (77)..(79): Xaa can be any amino acid
  MISC_FEATURE (77)..(79): Amino acids 77-79 may be present or absent
PKKKIQLHAEHALYDALMILNIXXXXXXXXXXXLEDYAFNFELILEEIARLFESGXXKDEAEKAKRMKEWMKRIKTXXXEDEQEEMANAIITILQSWIFS

SEQ ID NO: 10 - protein, 100 aa, Artificial sequence
  Synthetic construct
PKKKIQLHAEHALYDALMILNIVKTNSPPAEEKLEDYAFNFELILEEIACLFESGDQKDEAEKAKRMKEWMKRIKTTASEDEQEEMANAIITILQSWIFS

SEQ ID NO: 11 - protein, 100 aa, Artificial sequence
  Synthetic construct
  MISC_FEATURE (23)..(33): Xaa can be any amino acid
  MISC_FEATURE (23)..(33): Amino acids 23-33 may be present or absent
  MISC_FEATURE (56)..(57): Xaa can be any amino acid
  MISC_FEATURE (56)..(57): Amino acids 56-57 may be present or absent
  MISC_FEATURE (77)..(79): Xaa can be any amino acid
  MISC_FEATURE (77)..(79): Amino acids 77-79 may be present or absent
PKKKIQLHAEHALYDALMILNIXXXXXXXXXXXLEDYAFNFELILEEIACLFESGXXKDEAEKAKRMKEWMKRIKTXXXEDEQEEMANAIITILQSWIFS

SEQ ID NO: 12 - protein, 100 aa, Artificial sequence
  Synthetic construct
PKKKIQLHAEHALYDALMILNIVKTNSPPAEEKLEDYAFNFELILEEIARLFCSGDQKDEAEKAKRMKEWMKRIKTTASEDEQEEMANAIITILQSWIFS

SEQ ID NO: 13 - protein, 100 aa, Artificial sequence
  Synthetic construct
  MISC_FEATURE (23)..(33): Xaa can be any amino acid
  MISC_FEATURE (23)..(33): Amino acids 23-33 may be present or absent
  MISC_FEATURE (56)..(57): Xaa can be any amino acid
  MISC_FEATURE (56)..(57): Amino acids 56-57 may be present or absent
  MISC_FEATURE (77)..(79): Xaa can be any amino acid
  MISC_FEATURE (77)..(79): Amino acids 77-79 may be present or absent
PKKKIQLHAEHALYDALMILNIXXXXXXXXXXXLEDYAFNFELILEEIARLFCSGXXKDEAEKAKRMKEWMKRIKTXXXEDEQEEMANAIITILQSWIFS

SEQ ID NO: 14 - protein, 100 aa, Artificial sequence
  Synthetic construct
PKKKIQLHAEHALYDALMILNIVKTNSPPAEEKLEDYAFNFELILEEIARLFESGCQKDEAEKAKRMKEWMKRIKTTASEDEQEEMANAIITILQSWIFS

SEQ ID NO: 15 - protein, 100 aa, Artificial sequence
  Synthetic construct
  MISC_FEATURE (23)..(33): Xaa can be any amino acid
  MISC_FEATURE (23)..(33): Amino acids 23-33 may be present or absent
  MISC_FEATURE (77)..(79): Xaa can be any amino acid
  MISC_FEATURE (77)..(79): Amino acids 77-79 may be present or absent
PKKKIQLHAEHALYDALMILNIXXXXXXXXXXXLEDYAFNFELILEEIARLFESGCQKDEAEKAKRMKEWMKRIKTXXXEDEQEEMANAIITILQSWIFS

SEQ ID NO: 16 - protein, 100 aa, Artificial sequence
  Synthetic construct
PKKKIQLHAEHALYDALMILNIVKTNSPPAEEKLEDYAFNFELILEEIARLFESGDQCDEAEKAKRMKEWMKRIKTTASEDEQEEMANAIITILQSWIFS

SEQ ID NO: 17 - protein, 100 aa, Artificial sequence
  Synthetic construct
  MISC_FEATURE (23)..(33): Xaa can be any amino acid
  MISC_FEATURE (23)..(33): Amino acids 23-33 may be present or absent
  MISC_FEATURE (56)..(57): Xaa can be any amino acid
  MISC_FEATURE (56)..(57): Amino acids 56-57 may be present or absent
  MISC_FEATURE (77)..(79): Xaa can be any amino acid
  MISC_FEATURE (77)..(79): Amino acids 77-79 may be present or absent
PKKKIQLHAEHALYDALMILNIXXXXXXXXXXXLEDYAFNFELILEEIARLFESGXXCDEAEKAKRMKEWMKRIKTXXXEDEQEEMANAIITILQSWIFS

SEQ ID NO: 18 - protein, 100 aa, Artificial sequence
  Synthetic construct
PKKKIQLHAEHALYDALMILNIVKTNSPPAEEKLEDYAFNFELILEEIARLFESGDQKCEAEKAKRMKEWMKRIKTTASEDEQEEMANAIITILQSWIFS

SEQ ID NO: 19 - protein, 100 aa, Artificial sequence
  Synthetic construct
  MISC_FEATURE (23)..(33): Xaa can be any amino acid
  MISC_FEATURE (23)..(33): Amino acids 23-33 may be present or absent
  MISC_FEATURE (56)..(57): Xaa can be any amino acid
  MISC_FEATURE (56)..(57): Amino acids 56-57 may be present or absent
  MISC_FEATURE (77)..(79): Xaa can be any amino acid
  MISC_FEATURE (77)..(79): Amino acids 77-79 may be present or absent
PKKKIQLHAEHALYDALMILNIXXXXXXXXXXXLEDYAFNFELILEEIARLFESGXXKCEAEKAKRMKEWMKRIKTXXXEDEQEEMANAIITILQSWIFS

SEQ ID NO: 20 - protein, 100 aa, Artificial sequence
  Synthetic construct
PKKKIQLHAEHALYDALMILNIVKTNSPPAEEKLEDYAFNFELILEEIARLFESGDQKDEACKAKRMKEWMKRIKTTASEDEQEEMANAIITILQSWIFS

SEQ ID NO: 21 - protein, 100 aa, Artificial sequence
  Synthetic construct
  MISC_FEATURE (23)..(33): Xaa can be any amino acid
  MISC_FEATURE (23)..(33): Amino acids 23-33 may be present or absent
  MISC_FEATURE (56)..(57): Xaa can be any amino acid
  MISC_FEATURE (56)..(57): Amino acids 56-57 may be present or absent
  MISC_FEATURE (77)..(79): Xaa can be any amino acid
  MISC_FEATURE (77)..(79): Amino acids 77-79 may be present or absent
PKKKIQLHAEHALYDALMILNIXXXXXXXXXXXLEDYAFNFELILEEIARLFESGXXKDEACKAKRMKEWMKRIKTXXXEDEQEEMANAIITILQSWIFS

SEQ ID NO: 22 - protein, 100 aa, Artificial sequence
  Synthetic construct
PKKKIQLHAEHALYDALMILNIVKTNSPPAEEKLEDYAFNFELILEEIARLFESGDQKDEAEKAKCMKEWMKRIKTTASEDEQEEMANAIITILQSWIFS

SEQ ID NO: 23 - protein, 100 aa, Artificial sequence
  Synthetic construct
  MISC_FEATURE (23)..(33): Xaa can be any amino acid
  MISC_FEATURE (23)..(33): Amino acids 23-33 may be present or absent
  MISC_FEATURE (56)..(57): Xaa can be any amino acid
  MISC_FEATURE (56)..(57): Amino acids 56-57 may be present or absent
  MISC_FEATURE (77)..(79): Xaa can be any amino acid
  MISC_FEATURE (77)..(79): Amino acids 77-79 may be present or absent
PKKKIQLHAEHALYDALMILNIXXXXXXXXXXXLEDYAFNFELILEEIARLFESGXXKDEAEKAKCMKEWMKRIKTXXXEDEQEEMANAIITILQSWIFS

SEQ ID NO: 24 - protein, 100 aa, Artificial sequence
  Synthetic construct
PKKKIQLHAEHALYDALMILNIVKTNSPPAEEKLEDYAFNFELILEEIARLFESGDQKDEAEKAKRMKCWMKRIKTTASEDEQEEMANAIITILQSWIFS

SEQ ID NO: 25 - protein, 100 aa, Artificial sequence
  Synthetic construct
  MISC_FEATURE (23)..(33): Xaa can be any amino acid
  MISC_FEATURE (23)..(33): Amino acids 23-33 may be present or absent
  MISC_FEATURE (56)..(57): Xaa can be any amino acid
  MISC_FEATURE (56)..(57): Amino acids 56-57 may be present or absent
  MISC_FEATURE (77)..(79): Xaa can be any amino acid
  MISC_FEATURE (77)..(79): Amino acids 77-79 may be present or absent
PKKKIQLHAEHALYDALMILNIXXXXXXXXXXXLEDYAFNFELILEEIARLFESGXXKDEAEKAKRMKCWMKRIKTXXXEDEQEEMANAIITILQSWIFS

SEQ ID NO: 26 - protein, 100 aa, Artificial sequence
  Synthetic construct
PKKKIQLHAEHALYDALMILNIVKTNSPPAEEKLEDYAFNFELILEEIARLFESGDQKDEAEKAKRMKEWMKCIKTTASEDEQEEMANAIITILQSWIFS

SEQ ID NO: 27 - protein, 100 aa, Artificial sequence
  Synthetic construct
  MISC_FEATURE (23)..(33): Xaa can be any amino acid
  MISC_FEATURE (23)..(33): Amino acids 23-33 may be present or absent
  MISC_FEATURE (56)..(57): Xaa can be any amino acid
  MISC_FEATURE (56)..(57): Amino acids 56-57 may be present or absent
  MISC_FEATURE (77)..(79): Xaa can be any amino acid
  MISC_FEATURE (77)..(79): Amino acids 77-79 may be present or absent
PKKKIQLHAEHALYDALMILNIXXXXXXXXXXXLEDYAFNFELILEEIARLFESGXXKDEAEKAKRMKEWMKCIKTXXXEDEQEEMANAIITILQSWIFS

SEQ ID NO: 28 - protein, 100 aa, Artificial sequence
  Synthetic construct
PKKKIQLHAEHALYDALMILNIVKTNSPPAEEKLEDYAFNFELILEEIARLFESGDQKDEAEKAKRMKEWMKRIKTCASEDEQEEMANAIITILQSWIFS

SEQ ID NO: 29 - protein, 100 aa, Artificial sequence
  Synthetic construct
  MISC_FEATURE (23)..(33): Xaa can be any amino acid
  MISC_FEATURE (23)..(33): Amino acids 23-33 may be present or absent
  MISC_FEATURE (56)..(57): Xaa can be any amino acid
  MISC_FEATURE (56)..(57): Amino acids 56-57 may be present or absent
PKKKIQLHAEHALYDALMILNIXXXXXXXXXXXLEDYAFNFELILEEIARLFESGXXKDEAEKAKRMKEWMKRIKTCASEDEQEEMANAIITILQSWIFS

SEQ ID NO: 30 - protein, 100 aa, Artificial sequence
  Synthetic construct
PKKKIQLHAEHALYDALMILNIVKTNSPPAEEKLEDYAFNFELILEEIARLFESGDQKDEAEKAKRMKEWMKRIKTTASEDCQEEMANAIITILQSWIFS

SEQ ID NO: 31 - protein, 100 aa, Artificial sequence
  Synthetic construct
  MISC_FEATURE (23)..(33): Xaa can be any amino acid
  MISC_FEATURE (23)..(33): Amino acids 23-33 may be present or absent
  MISC_FEATURE (56)..(57): Xaa can be any amino acid
  MISC_FEATURE (56)..(57): Amino acids 56-57 may be present or absent
  MISC_FEATURE (77)..(79): Xaa can be any amino acid
  MISC_FEATURE (77)..(79): Amino acids 77-79 may be present or absent
PKKKIQLHAEHALYDALMILNIXXXXXXXXXXXLEDYAFNFELILEEIARLFESGXXKDEAEKAKRMKEWMKRIKTXXXEDCQEEMANAIITILQSWIFS

SEQ ID NO: 32 - protein, 100 aa, Artificial sequence
  Synthetic construct
PKKKIQLHAEHALYDALMILNIVKTNSPPAEEKLEDYAFNFELILEEIARLFESGDQKDEAEKAKRMKEWMKRIKTTASEDEQECMANAIITILQSWIFS

SEQ ID NO: 33 - protein, 100 aa, Artificial sequence
  Synthetic construct
  MISC_FEATURE (23)..(33): Xaa can be any amino acid
  MISC_FEATURE (23)..(33): Amino acids 23-33 may be present or absent
  MISC_FEATURE (56)..(57): Xaa can be any amino acid
  MISC_FEATURE (56)..(57): Amino acids 56-57 may be present or absent
  MISC_FEATURE (77)..(79): Xaa can be any amino acid
  MISC_FEATURE (77)..(79): Amino acids 77-79 may be present or absent
PKKKIQLHAEHALYDALMILNIXXXXXXXXXXXLEDYAFNFELILEEIARLFESGXXKDEAEKAKRMKEWMKRIKTXXXEDEQECMANAIITILQSWIFS

(19) United States
(12) Patent Application Publication
(10) Pub. No.: US 2022/0370563 A1
(43) Pub. Date: Nov. 24, 2022

(54) METHODS OF ADMINISTRATION OF IL-2 RECEPTOR AGONISTS

(71) Applicant: NEOLEUKIN THERAPEUTICS, INC., Seattle, WA (US)

(72) Inventors: Carl WALKEY, Seattle, WA (US); Jonathan DRACHMAN, Seattle, WA (US); Umut ULGE, Seattle, WA (US); Daniel Adriano SILVA MANZANO, Seattle, WA (US)

(21) Appl. No.: 17/771,192

(22) PCT Filed: Oct. 22, 2020
(86) PCT No.: PCT/US20/56846

§ 371 Date: Apr. 22, 2022

Related U.S. Application Data
(60) Provisional application No. 62/926,302, filed on Oct. 25, 2019.
(60) Provisional application No. 62/953,372, filed on Dec. 24, 2019.

(51) Int. Cl. A61K 38/20; A61K 47/60; A61K 47/54; A61K 47/68; A61K 9/00; A61P 35/00

(52) U.S. Cl. CPC A61K 38/2013; A61K 47/60; A61K 47/545; A61K 47/6813; A61K 9/0019; A61P 35/00

(57) ABSTRACT

The present invention provides, inter alia, treatment regimens for administration of an IL-2 receptor agonist. The present invention is directed to, inter alia, methods for modulating an immune response in a subject in need thereof with an IL-2 receptor agonist. In some aspects, the immune response is an anti-cancer immune response.

### CROSS-REFERENCES TO RELATED APPLICATIONS

This application claims priority to U.S. Provisional Patent Application Serial No. 62/926,302 filed Oct. 25, 2019 and 62/953,372 filed Dec. 24, 2019, each incorporated by reference herein in its entirety.

### REFERENCE TO A “SEQUENCE LISTING,” A TABLE, OR A COMPUTER PROGRAM LISTING APPENDIX SUBMITTED ON A COMPACT DISK

This application contains a Sequence Listing which has been submitted electronically in ASCII format and is hereby incorporated by reference in its entirety. Said ASCII copy, created on Oct. 21, 2020, is named 057318_501001WO_Sequence_Listing_ST25.txt and is 33.4 kilobytes in size.

### BACKGROUND OF THE INVENTION

The central immune cytokine interleukin 2 (IL-2) holds considerable potential for cancer treatment; IL2 immunotherapy is known, however, to be associated with various toxicities. Numerous efforts to improve its therapeutic properties by mutation and/or chemical modification and to develop new drugs with IL-2 agonist activity have been undertaken. There is an on-going need to develop optimal dosage regimens to help reduce toxicity and improve therapeutic effects associated with drugs having IL-2 agonist activity.

### BRIEF SUMMARY OF THE INVENTION

The present invention is directed to, inter alia, methods for modulating an immune response in a subject in need thereof with an IL-2 receptor agonist. In some aspects, the immune response is an anti-cancer immune response. In some aspects, the methods comprise the steps of (a) administering to the subject one or more priming doses of the IL-2 receptor agonist in order to enable escalation to a target dose level that would have an unacceptable tolerability profile if administered to the subject as a first dose, and (b) administering to the subject the IL-2 receptor agonist at the target dose level. In some aspects, the methods comprise administering to the subject in need thereof a dosing regimen of an IL-2 receptor agonist comprising administering to the subject one or more priming doses of the IL-2 receptor agonist at one or more priming dose levels following by administration of the IL-2 receptor agonist at a target dose level wherein the target dose level is greater than the priming dose levels. In exemplary embodiments, the IL-2 receptor agonist is a long-acting IL-2 receptor agonist. In exemplary embodiments, the IL-2 receptor agonist is a β [symbol] _{c} selective IL-2 receptor agonist.

The present invention is also directed to, inter alia, methods for treating cancer in a subject in need thereof with an IL-2 receptor agonist. In some aspects, the methods comprise the steps of (a) administering to the subject one or more priming doses of the IL-2 receptor agonist in order to enable escalation to a target dose level that would have an unacceptable tolerability profile if administered to the subject as a first dose, and (b) administering to the subject the IL-2 receptor agonist at the target dose level. In some aspects, the methods comprise administering to the subject in need thereof a dosing regimen of an IL-2 receptor agonist comprising administering to the subject one or more priming doses of the IL-2 receptor agonist at one or more priming dose levels following by administration of the IL-2 receptor agonist at a target dose level wherein the target dose level is greater than the priming dose levels. In exemplary embodiments, the IL-2 receptor agonist is a long-acting IL-2 receptor agonist. In exemplary embodiments, the IL-2 receptor agonist is a β [symbol] _{c} selective IL-2 receptor agonist.

The present invention is also directed to, inter alia, methods for (i) modulating an immune response or (ii) treating cancer in a subject in need thereof with a long-acting IL-2 receptor agonist comprising (i) selecting a target dose and a priming dose for administration to the subject; wherein the target dose is associated with an unacceptable tolerability profile if administered to the subject as a first dose but has a more favorable tolerability profile if administered after a priming dose (ii) administering one or more of the priming doses of the IL-2 receptor agonist to the subject in order to enable escalation to the target dose level, and administering to the subject the IL-2 receptor agonist at the target dose level.

The terms “dose” and “dose level” are used interchangeably throughout the application. As used herein, the term “subject” refers to an animal, preferably a mammal, more preferably a human. The terms “polypeptide”, “protein” or “peptide” refer to any chain of amino acid residues, regardless of its length or post-translational modification (e.g., glycosylation or phosphorylation). Microgram per kilogram is represented by ug/kg.

### BRIEF DESCRIPTION OF THE DRAWINGS

FIG. 1 shows the body-weight change of BALB/c mice implanted with CT-26 cells and receiving increasing doses of an IL-2 receptor agonist. Mice (N=8 per group) received vehicle or 150, 300, 600, or 1200 ug/kg of the IL-2 receptor agonist on day 10 of the study. A second dose was administered to surviving mice on Study Day 17. All mice receiving 150, 300, or 600 ug/kg of the test article survived both doses, while all mice receiving 1200 ug/kg were either found dead or moribund on Day 14, prior to the second dose. 600 ug/kg was determined to be the MTD.

FIG. 2 shows the body-weight change of BALB/c mice implanted with CT-26 cells and receiving a flat dosage regimen or step-dosing regimen of an IL-2 receptor agonist. The mice (N=10 per group) received either 500 ug/kg of the IL-2 receptor agonist on Day 9 and 500 ug/kg on Day 16 (flat dosage regimen) or 500 ug/kg of the IL-2 receptor agonist on Day 9 and 1000 ug/kg on Day 16. All mice receiving both 500 ug/kg and 1000 ug/kg survived the second dose.

### DETAILED DESCRIPTION OF THE INVENTION

The present invention provides, inter alia, treatment regimens for administration of an IL-2 receptor agonist. The present inventors have surprisingly discovered that a dose of an IL-2 receptor agonist that would be above the maximum tolerated dose (MTD) of the drug, if administered as a first dose, can be safely administered to the subject if the subject is first primed with one or more reduced doses of the drug. In other words, treatment with one or more priming doses enables escalation to a target dose that would not be tolerated if administered as a first dose. The priming dose primes the subject for administration of the target dose and each priming dose is always reduced as compared to the target dose. The one or more priming dose(s) initiates treatment with the IL-2 receptor agonist. Benefits of such a treatment regimen can include, for example, (i) improved efficacy of treatment as it allows for provision of a higher dose as compared to a regimen that keeps the dose constant throughout treatment and/or (ii) reduced treatment-associated toxicities as treatment initiation with a lower dose is associated with reduced toxicities as compared to treatment initiation with a higher dose. In some aspects, for maximum therapeutic effect, the priming dose level as well as the target dose level will be a therapeutically effective dose level.

### Long-Acting IL-2 Receptor Agonists

The term IL-2 receptor agonist as used herein refers to a polypeptide or protein capable of activating IL-2 receptor-mediated signaling. In particular preferred aspects, the IL-2 receptor agonist is an IL-2 receptor β [symbol] _{c} selective IL-2 receptor agonist. An IL-2 receptor β [symbol] _{c} selective IL-2 receptor agonist, as used herein, refers to an IL-2 receptor agonist that specifically binds to the IL-2 receptor β [symbol] _{c} but has reduced binding affinity to the IL-2 receptor alpha as compared to native IL-2. In some aspects, the IL-2 receptor agonist is alpha independent meaning it does not bind to the IL-2 receptor alpha. In some aspects, binding to IL-2 receptor alpha by an IL-2 receptor β [symbol] _{c} selective agonist of the present invention is decreased by at least 20 fold, 30 fold, or 50 fold as compared to native IL-2 whereas binding to the IL-2 receptor β [symbol] _{c} is increased, substantially the same, or decreased by no more than about 10 fold, preferably no more than about 5 fold as compared to the native IL-2. Methods of measuring binding affinities against IL-2 as a standard are known in the art.

In exemplary embodiments, the IL-2 receptor agonist is a long-acting IL-2 receptor agonist. By long-acting, it is meant that the agonist has a plasma or serum half-life of 3 hours or greater, preferably 4 hours or greater. In some aspects, the IL-2 receptor agonists will have a serum or plasma half-life of 9 or 10 hours or greater or 12 hours or greater. The half-life of a protein or polypeptide refers to the time necessary for the concentration of the polypeptide to decrease by 50% as measured by an appropriate assay. The reduction can be caused by in vivo degradation, clearance, or sequestration of the polypeptide. The half-life of an IL-2 receptor agonist can be determined by any manner known in the art in view of the present disclosure, such as by measuring the concentration of the IL-2 receptor agonist in the blood. For example, to measure the half-life of a protein or polypeptide in vivo, a suitable dose of the protein or polypeptide is administered to a warm-blooded animal (i.e. to a human or to another suitable mammal, such as a mouse, rabbit, rat, pig, dog, or a primate); blood samples or other samples from the animal are collected; the level or concentration of the protein or polypeptide in the sample is determined; and the time until the level or concentration of the polypeptide has been reduced by 50% is calculated based on measured data. See, e.g., Kenneth, A et al., Chemical Half-life of Pharmaceuticals: A Handbook for Pharmacists and Peters et al., Pharmacokinetic analysis: A Practical Approach (1996). As used herein, “an increase in half-life” or “longer half-life” refers to an increase in any one or more of the parameters used to describe the protein half-life, such as the t½-alpha, t½-beta and the area under the curve (AUC), as compared to a control. The long-acting nature of the IL-2 receptor agonist can be due to a moiety that it is conjugated or fused to the IL-2 polypeptide.

In some aspects, the IL-2 receptor agonist is an IL-2 polypeptide. Within the context of this invention, the term “IL-2” designates any source of IL-2, including mammalian sources and may be native or obtained by recombinant or synthetic techniques, including recombinant IL-2 polypeptides produced by microbial hosts. The term “IL-2” includes polypeptides comprising the native polypeptide sequence as well as active variants of the native IL-2 polypeptide. In some exemplary aspects, the IL-2 polypeptide, including an active variant thereof, is derived from a human source, and includes recombinant human IL-2, particularly recombinant human IL-2 produced by microbial hosts. Active variants of IL-2 are known in the art. Variants of the native IL-2 can be fragments, analogues, and derivatives thereof. By “fragment” is intended a polypeptide comprising only a part of the intact polypeptide sequence. An “analogue” designates a polypeptide comprising the native polypeptide sequence with one or more amino acid substitutions, insertions, or deletions. The amino acid substitution or insertion can be a natural amino acid or can be an unnatural amino acid. As used herein, the natural amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V). As used herein, the term “unnatural amino acid” refers to an amino acid other than the 20 amino acids that occur naturally in protein. Unnatural amino acids are known in the art. “Derivatives” include any modified native IL-2 polypeptide or fragment or analogue thereof, including, for example, glycosylated, phosphorylated, fused to another polypeptide or molecule, or polymerized. Active variants of a reference IL-2 polypeptide generally have at least 75%, preferably at least 85%, more preferably at least 90% amino acid sequence identity to the amino acid sequence of the reference IL-2 polypeptide (e.g., human IL-2 sequence).

Methods for determining whether a variant IL-2 polypeptide is active are known in the art, e.g., via a STATS phosphorylation assay. An amino acid sequence of human IL-2 is disclosed, for example, in Genbank ref P60568.

Exemplary IL-2 receptor agonists of the present invention include IL-2 mimetics. IL-2 mimetics are described in Silva et al., Nature 2019 Jan;565(7738):186-191. Exemplary IL-2 mimetics to be used in the present methods induce heterodimerization of IL-2Rβ [symbol] _{c}, leading to phosphorylation of STATS. IL-2 mimetics of the present invention bind to the IL-2 receptor β [symbol] _{c} heterodimer (IL-2Rβ [symbol] _{c}) and, in some aspects, are non-naturally occurring polypeptides comprising four helical peptides, X1, X2, X3, and X4. X1, X2, X3, and X4 are also referred to herein as domains. X1, X2, X3, and X4 may be in any order in the polypeptide and amino acid linkers may be present between any of the domains. The amino acid linkers may be of any length as deemed appropriate for an intended use. Exemplary lengths of amino acids include linkers between 1-200, 1-100, 1-50, 1-20, 1-15, 1-10, 2-20, 2-15, or 2-10 amino acids in length. As with the variability permitted in the amino acid residues and flexibility in domain order, the flexibility in linker stems from the use of de novo protein design to construct the IL-2 mimetics. In these mimetics, the majority of the contributions to protein folding come from hydrophobic core interactions among the secondary structure elements rather than from the linkers. At least for that reason, the linkers can generally be modified without compromising protein folding.

-2 receptor agonists of the present invention include IL-2 mimetics wherein X1 is a peptide comprising an amino acid sequence at least 25%, 27%, 30%, 35%, 40%, 45%, 50%, 55%, 60%, 65%, 70%, 75%, 80%, 85%, 90%, 95%, 98%, or 100% identical to EHALYDAL (SEQ ID NO:1); X2 is a helical-peptide of at least 8 amino acids in length; X3 is a peptide comprising an amino acid sequence at least 25%%, 27%, 30%, 35%, 40%, 45%, 50%, 55%, 60%, 65%, 70%, 75%, 80%, 85%, 90%, 95%, 98%, or 100% identical to YAFNFELI (SEQ ID NO:2); and X4 is a peptide comprising an amino acid sequence at least 25%, 27%, 30%, 35%, 40%, 45%, 50%, 55%, 60%, 65%, 70%, 75%, 80%, 85%, 90%, 95%, 98%, or 100% identical to ITILQSWIF (SEQ ID NO:3).

IL-2 receptor agonists of the present invention include IL-2 mimetics wherein X1 is a peptide comprising an amino acid sequence at least 60% identical to EHALYDAL (SEQ ID NO:1); X2 is a helical-peptide of at least 8 amino acids in length; X3 is a peptide comprising an amino acid sequence at least 60% identical to YAFNFELI (SEQ ID NO:2); and X4 is a peptide comprising an amino acid sequence at least 60% identical to ITILQSWIF (SEQ ID NO:3).

IL-2 receptor agonists of the present invention include IL-2 mimetics wherein X1 is a peptide comprising an amino acid sequence at least 75% identical to EHALYDAL (SEQ ID NO:1); X2 is a helical-peptide of at least 8 amino acids in length; X3 is a peptide comprising an amino acid sequence at least 75% identical to YAFNFELI (SEQ ID NO:2); and X4 is a peptide comprising an amino acid sequence at least 75% identical to ITILQSWIF (SEQ ID NO:3).

IL-2 receptor agonists of the present invention include IL-2 mimetics wherein X1 is a peptide comprising an amino acid sequence at least 85% identical to EHALYDAL (SEQ ID NO:1); X2 is a helical-peptide of at least 8 amino acids in length; X3 is a peptide comprising an amino acid sequence at least 85% identical to YAFNFELI (SEQ ID NO:2); and X4 is a peptide comprising an amino acid sequence at least 85% identical to ITILQSWIF (SEQ ID NO:3).

X1, X3, and X4 may be of any suitable length, meaning each domain may contain any suitable number of additional amino acids in addition to the amino acids of SEQ ID NOS:1, 2, and 3, respectively. Typically, each of X1, X3 and X4 comprise at least 8 amino acids. In some aspects, each of X1, X3 and X4 comprise at least 21 amino acids. In some such aspects, each of X1, X3 and X4 is no more than 200 or 100 or 50 amino acids in length

IL-2 receptor agonists of the present invention include IL-2 mimetics wherein X1 is a peptide comprising an amino acid sequence at least 25%, 27%, 30%, 35%, 40%, 45%, 50%, 55%, 60%, 65%, 70%, 75%, 80%, 85%, 90%, 95%, 98%, or 100% identical along its length to the peptide PKKKIQLHAEHALYDALMILNI (SEQ ID NO: 4), X2 is a helical-peptide of at least 8 amino acids in length; X3 is a peptide comprising an amino acid sequence at least 25%, 27%, 30%, 35%, 40%, 45%, 50%, 55%, 60%, 65%, 70%, 75%, 80%, 85%, 90%, 95%, 98%, or 100% identical along its length the peptide LEDYAFNFELILEEIARLFESG (SEQ ID NO:5); and X4 is a peptide comprising an amino acid sequence at least 25%, 27%, 30%, 35%, 40%, 45%, 50%, 55%, 60%, 65%, 70%, 75%, 80%, 85%, 90%, 95%, 98%, or 100% identical along its length to the peptide EDEQEEMANAIITILQSWIFS(SEQ ID NO:6).

IL-2 receptor agonists of the present invention include IL-2 mimetics wherein X1 is a peptide comprising an amino acid sequence at least 70% identical along its length to the peptide PKKKIQLHAEHALYDALMILNI (SEQ ID NO: 4), X2 is a helical-peptide of at least 8 amino acids in length; X3 is a peptide comprising an amino acid sequence at least 70% identical along its length the peptide LEDYAFNFELILEEIARLFESG (SEQ ID NO:5); and X4 is a peptide comprising an amino acid sequence at least 70% identical along its length to the peptide EDEQEEMANAIITILQSWIFS(SEQ ID NO:6).

IL-2 receptor agonists of the present invention include IL-2 mimetics wherein X1 is a peptide comprising an amino acid sequence at least 75% identical along its length to the peptide PKKKIQLHAEHALYDALMILNI (SEQ ID NO: 4), X2 is a helical-peptide of at least 8 amino acids in length; X3 is a peptide comprising an amino acid sequence at least 75% identical along its length the peptide LEDYAFNFELILEEIARLFESG (SEQ ID NO:5); and X4 is a peptide comprising an amino acid sequence at least 75% identical along its length to the peptide EDEQEEMANAIITILQSWIFS(SEQ ID NO:6).

IL-2 receptor agonists of the present invention include IL-2 mimetics wherein X1 is a peptide comprising an amino acid sequence at least 80% identical along its length to the peptide PKKKIQLHAEHALYDALMILNI (SEQ ID NO: 4), X2 is a helical-peptide of at least 8 amino acids in length; X3 is a peptide comprising an amino acid sequence at least 80% identical along its length the peptide LEDYAFNFELILEEIARLFESG (SEQ ID NO:5); and X4 is a peptide comprising an amino acid sequence at least 80% identical along its length to the peptide EDEQEEMANAIITILQSWIFS(SEQ ID NO:6).

IL-2 receptor agonists of the present invention include IL-2 mimetics wherein X1 is a peptide comprising an amino acid sequence at least 85% identical along its length to the peptide PKKKIQLHAEHALYDALMILNI (SEQ ID NO: 4), X2 is a helical-peptide of at least 8 amino acids in length; X3 is a peptide comprising an amino acid sequence at least 85% identical along its length the peptide LEDYAFNFELILEEIARLFESG (SEQ ID NO:5); and X4 is a peptide comprising an amino acid sequence at least 85% identical along its length to the peptide EDEQEEMANAIITILQSWIFS(SEQ ID NO:6).

IL-2 receptor agonists of the present invention include IL-2 mimetics wherein X1 is a peptide comprising an amino acid sequence at least 90% identical along its length to the peptide PKKKIQLHAEHALYDALMILNI (SEQ ID NO: 4), X2 is a helical-peptide of at least 8 amino acids in length; X3 is a peptide comprising an amino acid sequence at least 90% identical along its length the peptide LEDYAFNFELILEEIARLFESG (SEQ ID NO:5); and X4 is a peptide comprising an amino acid sequence at least 90% identical along its length to the peptide EDEQEEMANAIITILQSWIFS(SEQ ID NO:6).

IL-2 receptor agonists of the present invention include IL-2 mimetics wherein X1 is a peptide comprising an amino acid sequence at least 95% identical along its length to the peptide PKKKIQLHAEHALYDALMILNI (SEQ ID NO: 4), X2 is a helical-peptide of at least 8 amino acids in length; X3 is a peptide comprising an amino acid sequence at least 95% identical along its length the peptide LEDYAFNFELILEEIARLFESG (SEQ ID NO:5); and X4 is a peptide comprising an amino acid sequence at least 95% identical along its length to the peptide EDEQEEMANAIITILQSWIFS(SEQ ID NO:6).

IL-2 receptor agonists of the present invention include IL-2 mimetics wherein X1 is a peptide comprising the amino acid sequence PKKKIQLHAEHALYDALMILNI (SEQ ID NO: 4), X2 is a helical-peptide of at least 8 amino acids in length; X3 is a peptide comprising the amino acid sequence LEDYAFNFELILEEIARLFESG (SEQ ID NO:5); and X4 is a peptide comprising the amino acid sequence EDEQEEMANAIITILQSWIFS(SEQ ID NO:6). For all of these IL-2 mimetics, X1, X2, X3, and X4 may be in any order in the polypeptide. An exemplary order of domains is X1-X3-X2-X4.

In some such aspects for the IL-2 mimetics wherein X1 is a peptide comprising an amino acid sequence at least 25%, 27%, 30%, 35%, 40%, 45%, 50%, 55%, 60%, 65%, 70%, 75%, 80%, 85%, 90%, 95%, 98%, or 100% identical along its length to the peptide PKKKIQLHAEHALYDALMILNI (SEQ ID NO: 4); X2 is a helical-peptide of at least 8 amino acids in length; X3 is a peptide comprising an amino acid sequence at least 25%, 27%, 30%, 35%, 40%, 45%, 50%, 55%, 60%, 65%, 70%, 75%, 80%, 85%, 90%, 95%, 98%, or 100% identical along its length the peptide LEDYAFNFELILEEIARLFESG (SEQ ID NO:5); and X4 is a peptide comprising an amino acid sequence at least 25%, 27%, 30%, 35%, 40%, 45%, 50%, 55%, 60%, 65%, 70%, 75%, 80%, 85%, 90%, 95%, 98%, or 100% identical along its length to the peptide EDEQEEMANAIITILQSWIFS(SEQ ID NO:6); X1 includes 1, 2, 3, 4, or all 5 of the following: L at residue 7, H at residue 8, H at residue 11, Y at residue 14; M at residue 18; and/or (ii) X3 includes 1, 2, 3, 4, 5, 6, 7, or all 8 of the following: D at residue 3, Y at residue 4, F at residue 6, N at residue 7, L at residue 10, I at residue 11, E at residue 13, or E at residue 14. In a further embodiment, (iii) X4 includes I at residue 19. The noted positions for X1 are numbered in reference to SEQ ID NO:4; the noted positions for X3 are numbered in reference to SEQ ID NO:5; and the noted positions for X4 are numbered in reference to SEQ ID NO:6.

In some such aspects for the IL-2 mimetics, amino acid substitutions relative to SEQ ID NO:1 do not occur at positions 1E, 4L, 5Y, 6D, and 8L; amino acid substitutions relative to SEQ ID NO:2 do not occur at positions 1Y, 4N, 7L, and 81; amino acid substitutions relative to SEQ ID NO:3 do not occur at positions 1I, 5Q, and 7W; amino acid substitutions relative to SEQ ID NO:4 do not occur at positions 10E, 13L, 14Y, 15D, and 17L; amino acid substitutions relative to SEQ ID NO:5 do not occur at positions 1L, 4Y, 7N, 10L, 11I and 151; amino acid substitutions relative to SEQ ID NO:6 do not occur at positions 121, 16Q, and 18W.

IL-2 receptor agonists of the present invention include IL-2 mimetics wherein X1 is a peptide comprising an amino acid sequence at least 25%, 27%, 30%, 35%, 40%, 45%, 50%, 55%, 60%, 65%, 70%, 75%, 80%, 85%, 90%, 95%, 98%, or 100% identical along its length to the peptide PKKKIQLHAEHALYDALMILNI (SEQ ID NO: 4); X2 is a helical-peptide of at least 8 amino acids in length; X3 is a peptide comprising an amino acid sequence at least 25%, 27%, 30%, 35%, 40%, 45%, 50%, 55%, 60%, 65%, 70%, 75%, 80%, 85%, 90%, 95%, 98%, or 100% identical along its length the peptide

LEDYAFNFELILEEIARLFESG (SEQ ID NO:5); and X4 is a peptide comprising an amino acid sequence at least 25%, 27%, 30%, 35%, 40%, 45%, 50%, 55%, 60%, 65%, 70%, 75%, 80%, 85%, 90%, 95%, 98%, or 100% identical along its length to the peptide EDEQEEMANAIITILQSWIFS(SEQ ID NO:6); wherein an amino acid sequence at least 25%, 27%, 30%, 35%, 40%, 45%, 50%, 55%, 60%, 65%, 70%, 75%, 80%, 85%, 90%, 95%, 98%, or 100% identical to EHALYDAL (SEQ ID NO:1) is comprised within SEQ ID NO:4; an amino acid sequence at least 25%%, 27%, 30%, 35%, 40%, 45%, 50%, 55%, 60%, 65%, 70%, 75%, 80%, 85%, 90%, 95%, 98%, or 100% identical to YAFNFELI (SEQ ID NO:2) is comprising within SEQ ID NO:5; and an amino acid sequence at least 25%, 27%, 30%, 35%, 40%, 45%, 50%, 55%, 60%, 65%, 70%, 75%, 80%, 85%, 90%, 95%, 98%, or 100% identical to ITILQSWIF (SEQ ID NO:3) is comprised within SEQ ID NO:6.

IL-2 receptor agonists of the present invention include IL-2 mimetics wherein X1 is a peptide comprising an amino acid sequence at least 25%, 27%, 30%, 35%, 40%, 45%, 50%, 55%, 60%, 65%, 70%, 75%, 80%, 85%, 90%, 95%, 98%, or 100% identical along its length to the peptide PKKKIQLHAEHALYDALMILNI (SEQ ID NO: 4); X2 is a helical-peptide of at least 8 amino acids in length; X3 is a peptide comprising an amino acid sequence at least 25%, 27%, 30%, 35%, 40%, 45%, 50%, 55%, 60%, 65%, 70%, 75%, 80%, 85%, 90%, 95%, 98%, or 100% identical along its length the peptide LEDYAFNFELILEEIARLFESG (SEQ ID NO:5); and X4 is a peptide comprising an amino acid sequence at least 25%, 27%, 30%, 35%, 40%, 45%, 50%, 55%, 60%, 65%, 70%, 75%, 80%, 85%, 90%, 95%, 98%, or 100% identical along its length to the peptide EDEQEEMANAIITILQSWIFS(SEQ ID NO:6); wherein an amino acid sequence at least 85% identical to EHALYDAL (SEQ ID NO:1) is comprised within SEQ ID NO:4; an amino acid sequence at least 85% identical to YAFNFELI (SEQ ID NO:2) is comprising within SEQ ID NO:5; and an amino acid sequence at least 85% identical to ITILQSWIF (SEQ ID NO:3) is comprised within SEQ ID NO:6.

Identity as used herein in reference to polypeptide sequences, refers to the amino acid sequence identity between two molecules. When an amino acid position in both molecules is occupied by the same amino acid, then the molecules are identical at that position. The identity between two polypeptides is a direct function of the number of identical positions. In general, the sequences are aligned so that the highest order match is obtained (including gaps if necessary). Identity can be calculated using published techniques and widely available computer programs, such as the GCG program package (Devereux et al., Nucleic Acids Res. 12:387, 1984), BLASTP, FASTA (Atschul et al., J. Molecular Biol. 215:403, 1990), etc. Sequence identity can be measured, for example, using sequence analysis software such as the Sequence Analysis Software Package of the Genetics Computer Group at the University of Wisconsin Biotechnology Center (1710 University Avenue, Madison, Wis. 53705), with the default parameters thereof. When determining identity for the present invention, it is also important to consider positioning of the binding interfaces with the IL-2 receptor. If amino acids are added or deleted, it should be done in such a way that doesn't substantially interfere with presentation of the protein to its binding partner and with secondary structure. Generally, but not necessarily, it is preferable for amino acid substitutions relative to the reference peptide domains to be conservative amino acid substitutions. As used herein, “conservative amino acid substitution” means a given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are known. Polypeptides comprising conservative amino acid substitutions can be tested in any one of the assays described herein to confirm that a desired activity, e.g. antigen-binding activity and specificity of a native or reference polypeptide is retained. Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into H is; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu. In some aspects, an amino acid that is not necessary for binding or activity is replaced by cysteine to allow for attachment of a desirable moiety.

IL-2 receptor agonists of the present invention include IL-2 mimetics wherein X1 is a peptide comprising an amino acid sequence at least 25%, 27%, 30%, 35%, 40%, 45%, 50%, 55%, 60%, 65%, 70%, 75%, 80%, 85%, 90%, 95%, 98%, or 100% identical along its length to the peptide PKKKIQLHAEHALYDALMILNI (SEQ ID NO: 4) wherein

the amino acid at position 1 is P or if substituted is A, F, I, L, M, Q, R, S, or W;
the amino acid at position 2 is K or if substituted is A, D, E, G, or V;
the amino acid at position 3 is K or if substituted is D, E, F, or W;
the amino acid at position 4 is K or if substituted is D, E, N, P, R, or W;
the amino acid at position 5 is I or if substituted is D, E, H, K, L, M, or S;
the amino acid at position 6 is Q or if substituted is A, D, E, G, L, P, S, or W;
the amino acid at position 7 is L or if substituted is D, E, Q, Y, or I;
the amino acid at position 8 is H or if substituted is A, F, W, Y, M, or T;
the amino acid at position 9 is A or if substituted is C, F, or P;
the amino acid at position 10 is E or if substituted is C, D, F, K, or P;
the amino acid at position 11 is H or if substituted is D, F, or E;
the amino acid at position 12 is A or if substituted is D, E, P, S, T, or V;
the amino acid at position 13 is L or if substituted is H, I, M, P, R, V, or W;
the amino acid at position 14 is Y or if substituted is F, R, W, or K;
the amino acid at position 15 is D or if substituted is E, N, or Y;
the amino acid at position 16 is A or if substituted is C, L, M, or S;
the amino acid at position 17 is L or if substituted is F, I, M, P, or R;
the amino acid at position 18 is M or if substituted is G, Q, Y, or S;
the amino acid at position 19 is I or if substituted is L, M, P, Q, or V;
the amino acid at position 20 is L or if substituted is A, K, M, Q, R, or S;
the amino acid at position 21 is N or if substituted is G, K, P, R, S, or W;
the amino acid at position 22 is I or if substituted is D, E, K, M, N, W, or Y

In some such embodiments, 1, 2, 3, 4, or 5 of the following are not true: position 7 is I, position 8 is M or T, position 11 is E, position 14 is K, and position 18 is S.

IL-2 receptor agonists of the present invention include IL-2 mimetics wherein X3 is a peptide comprising an amino acid sequence at least 25%, 27%, 30%, 35%, 40%, 45%, 50%, 55%, 60%, 65%, 70%, 75%, 80%, 85%, 90%, 95%, 98%, or 100% identical along its length the peptide LEDYAFNFELILEEIARLFESG (SEQ ID NO:5) wherein:

the amino acid at position 1 is L or if substituted is A;
the amino acid at position 2 is E or if substituted is D, G, K, M, or T;
the amino acid at position 3 is D or if substituted is E, N, Y, or R;
the amino acid at position 4 is Y or if substituted is C, D, G, T, or F;
the amino acid at position 5 is A or if substituted is F, H, S, V, W, or Y;
the amino acid at position 6 is F or if substituted is A, I, M, T, V, Y, or K;
the amino acid at position 7 is N or if substituted is D, K, S, T, or R;
the amino acid at position 8 is F or if substituted is A, C, G, L, M, S, or V;
the amino acid at position 9 is E or if substituted is C, H, K, L, R, S, T, or V;
the amino acid at position 10 is L or if substituted is F, I, M, Y, or R;
the amino acid at position 11 is I or if substituted is L, N, T, or Y;
the amino acid at position 12 is L or if substituted is F, K, M, S, or V;
the amino acid at position 13 is E or if substituted is A, D, F, G, I, N, P, Q, S, T, or W;
the amino acid at position 14 is E or if substituted is A, F, G, H, S, or V;
the amino acid at position 15 is I or if substituted is C, L, M, V, or W;
the amino acid at position 16 is A or if substituted is D, G, S, T, or V;
the amino acid at position 17 is R or if substituted is H, K, L, or N;
the amino acid at position 18 is L or if substituted is C, D, G, I, Q, R, T, or W;
the amino acid at position 19 is F or if substituted is D, M, N, or W;
the amino acid at position 20 is E or if substituted is A, C, F, G, M, S, or Y;
the amino acid at position 21 is S or if substituted is D, E, G, H, L, M, R, T, V, or W;
the amino acid at position 22 is G or if substituted is A, D, K, N, S, or Y

In some such embodiments, 1, 2, 3, 4, 5, 6, 7, or all 8 of the following are not true: position 3 is R, position 4 is F, position 6 is K, position 7 is R, position 10 is R, position 11 is N, position 13 is W, and position 14 is G.

IL-2 receptor agonists of the present invention include IL-2 mimetics wherein X4 is a peptide comprising an amino acid sequence at least 25%, 27%, 30%, 35%, 40%, 45%, 50%, 55%, 60%, 65%, 70%, 75%, 80%, 85%, 90%, 95%, 98%, or 100% identical along its length to the peptide EDEQEEMANAIITILQSWIFS(SEQ ID NO:6) wherein:

the amino acid at position 1 is E or if substituted is D, G, K, or V;
the amino acid at position 2 is D or if substituted is I, M, or S;
the amino acid at position 3 is E or if substituted is G, H, or K;
the amino acid at position 4 is Q or if substituted is E, G, I, K, R, or S;
the amino acid at position 5 is E or if substituted is A, D, G, H, S, or V;
the amino acid at position 6 is E or if substituted is C, D, G, I, M, Q, R, T, or V;
the amino acid at position 7 is M or if substituted is C, E, L, P, R, or T;
the amino acid at position 8 is A or if substituted is F, L, M, or W;
the amino acid at position 9 is N or if substituted is A, G, L, Q, R, or T;
the amino acid at position 10 is A or if substituted is C, D, E, F, H, I, or W;
the amino acid at position 11 is I or if substituted is M, N, S, V, or W;
the amino acid at position 12 is I or if substituted is K, L, S, or V;
the amino acid at position 13 is T or if substituted is C, L, M, R, or S;
the amino acid at position 14 is I or if substituted is L, P, T, or Y;
the amino acid at position 15 is L or if substituted is F, G, I, M, N, or V;
the amino acid at position 16 is Q or if substituted is H, K, or R;
the amino acid at position 17 is S or if substituted is C, F, K, W, or Y;
the amino acid at position 18 is W or if substituted is K, Q, or T;
the amino acid at position 19 is I or if substituted is C, G, or N;
the amino acid at position 20 is F or if substituted is C, G, L, or Y; and
the amino acid at position 21 is S or if substituted is A, F, G, H, or Y.

In some such embodiments, position 19 is not I. In some such aspects, position 19 is C, G, or N.

As noted herein, domain X2 is a structural domain, and thus any amino acid sequence that connects the relevant other domains (depending on domain order) and allows them to fold can be used. The length required will depend on the structure of the protein being made and can be 8 amino acids or longer and in some aspects is 19 amino acids or longer. In some such aspects, X2 is no more than 200 or 100 or 50 amino acids in length. In any of the embodiments provided herein for the IL-2 mimetics, X2 can be a peptide comprising an amino acid sequence at least 20%, 27%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identical along its length to KDEAEKAKRMKEWMKRIKT (SEQ ID NO:7).

IL-2 receptor agonists of the present invention include IL-2 mimetics wherein X2 is a peptide comprising the amino acid sequence at least 20%, 27%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identical along its length to KDEAEKAKRMKEWMKRIKT (SEQ ID NO:7) wherein:

the amino acid at position 1 is K or if substituted is A, H, L, M, R, S, or V;
the amino acid at position 2 is D or if substituted is A, E, Q, R, S, T, V, W, or Y;
the amino acid at position 3 is E or if substituted is C, G, K, L, N, Q, R, or W;
the amino acid at position 4 is A or if substituted is F, G, N, S, T, V, or Y;
the amino acid at position 5 is E or if substituted is A, G, I, M, R, V, or C;
the amino acid at position 6 is K or if substituted is C, E, L, N, R, or V;
the amino acid at position 7 is A or if substituted is C, E, I, L, S, T, V, or W;
the amino acid at position 8 is K or if substituted is H, L, M, S, T, W, or Y;
the amino acid at position 9 is R or if substituted is A, I, L, M, Q, or S;
the amino acid at position 10 is M or if substituted is A, I, S, W, or Y;
the amino acid at position 11 is K or if substituted is C, I, L, S, or V;
the amino acid at position 12 is E or if substituted is C, K, L, P, Q, R, or T;
the amino acid at position 13 is W or if substituted is A, D, H, or N;
the amino acid at position 14 is M or if substituted is A, C, G, I, L, S, T, or V;
the amino acid at position 15 is K or if substituted is A, E, G, I, L, M, R, or V;
the amino acid at position 16 is R or if substituted is G, H, L, S, T, V, or C;
the amino acid at position 17 is I or if substituted is A, L, or V;
the amino acid at position 18 is K or if substituted is A, C, D, E, G, H, I, M, or S; and
the amino acid at position is 19 is T or if substituted is D, E, G, L, N, or V

An exemplary IL-2 receptor agonist of the present invention is an IL-2 mimetic comprising a polypeptide at least 25%, 27%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identical along its length to the amino acid sequence set forth in SEQ ID NO:8 or SEQ ID NO:9. SEQ ID NO:8 is the amino acid sequence of the Neo-2/15 protein described in Silva et al., Nature 2019 Jan;565(7738):186-191. SEQ ID NO:9 is the identical sequence to SEQ ID NO:8 except that the linker amino acids are optional and each amino acid residue of the linker, when present, may comprise any natural or unnatural amino acid. In some aspects, the amino acid linkers comprise all natural amino acids. In some aspects, the amino acid linker comprises one or more unnatural amino acid (e.g., from 1 to 3, 1 to 2, or 1 unnatural amino acid). The linker amino acids are denoted by the label X and are underlined. In exemplary embodiments, the amino acids are natural amino acids. The amino acid linkers, when present, connect the domains. The amino acid linkers may be of any length as deemed appropriate for an intended use.

| | |
|---|---|
| SEQ ID NO: 8 | PKKKIQLHAEHALYDALMILNIVKTNSPPAEEKLEDYAFNFELILEEIARL |
| (NEO 2-15): | FESGDQKDEAEKAKRMKEWMKRIKTTASEDEQEEMANAIITILQSWIFS |
| SEQ ID NO: 9 | PKKKIQLHAEHALYDALMILNIXXXXXXXXXXXLEDYAFNFELILEEIARL |
| | FESGXXKDEAEKAKRMKEWMKRIKTXXXEDEQEEMANAIITILQSWIFS |

IL-2 receptor agonists of the present invention include IL-2 mimetics comprising a polypeptide at least 25%, 27%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identical along its length to the amino acid sequence selected from SEQ ID NO:8 or 9 wherein 1, 2, 3, 4, 5, 6, 7, 8, 9, 10, 11, 12, 13, or all 14 of the following are not true: position 7 is I, position 8 is T or M, position 11 is E, position 14 is K, position 18 is S, position 33 is Q, position 36 is R, position 37 is F, position 39 is K, position 40 is R, position 43 is R, position 44 is N, position 46 is W, and position 47 is G. In a further embodiment, one or both of the following are not true: position 68 is I and position 98 is F. The skilled practitioner will understand that the above referenced positions are with reference to SEQ ID NO: 8. For SEQ ID NO: 9, when the length of the linkers separating the helical domains are of different length as compare to SEQ ID NO:8, the numbering of residues will change accordingly. Thus, for SEQ ID NO:9, reference to position 7 means the position in SEQ ID NO:9 corresponding to position 7 in SEQ ID NO: 8.

Cysteine residues in the IL-2 mimetics described herein can be used for attachment of a moiety (e.g., a stability moiety such as, for example, a water stabilizing moiety such as a PEG-containing moiety) to the polypeptide. The cysteine moiety can be in any one of X1, X2, X3, or X4 or optional linker. In some aspects, the cysteine moiety is in X2. For example, an exemplary IL-2 receptor agonist of the present invention is a Neo-2/15 polypeptide wherein an amino acid of Neo-2/15 is mutated to a cysteine residue for attachment of a moiety (e.g., a stability moiety such as, for example, a water stabilizing moiety such as a PEG-containing moiety) thereto. In some aspects, an exemplary IL-2 receptor agonist of the present invention is a Neo-2/15 polypeptide and an amino acid at one or more of positions 50, 53, 62, 69, 73, 82, 56, 58, 59, 66, 77, or 85 relative to SEQ ID NO:8 is mutated to a cysteine residue for attachment of a moiety (e.g., PEG-containing moiety) thereto.

Accordingly, in a further embodiment, an exemplary IL-2 receptor agonist of the present invention comprises a polypeptide at least 25%, 27%, 30%, 35%, 40%, 45%, 50%, 55%, 60%, 65%, 70%, 75%, 80%, 85%, 90%, 95%, 98%, or 100% identical to the full length of the amino acid sequence of SEQ ID NO:8 or 9 but for one, two, three, four, five, six, seven, eight, nine, ten, eleven, or all twelve of the following mutations are present (numbering is in reference to SEQ ID NO:8):

D56C;

K58C;

D59C;

R66C;

T77C;

E85C;

R50C;

E53C;

E62C;

E69C;

R73C; and/or

E82C.

Exemplary IL-2 receptor agonists of the present invention include IL-2 mimetics comprising a polypeptide at least 25%, 27%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identical along its length to an amino acid sequence set forth in any one of SEQ ID NOs:10-33. Underlined residues are linkers and are optional and each residue of the linker, when present, may comprise any amino acid. For each variant below, two SEQ ID NOS are provided: a first SEQ ID NO: that lists the sequence with the linker designated and a second SEQ ID NO: that includes the linker positions as optional and variable (similar to the format of SEQ ID NO:9). The linker amino acids are denoted by the label X and are underlined. In exemplary embodiments, the amino acids are natural amino acids. It is understood that the position equivalent to R50C (or the listed mutation) will be incorporated in the specified location depending on the length of the linker. The teachings provided herein, including those with respect to the X1, X2, X3, and X4 domains, along with the skill in the art, can be used to make IL-2 mimetics that comprise variants of the amino acid sequences set forth in SEQ ID NOs:10-33 for use as IL-2 receptor agonists in the present methods.

TABLE A

| | | |
|---|---|---|
| R50C | SEQ ID | PKKKIQLHAEHALYDALMILNIVKTNSPPAEEKLEDYAFNFELILEEIACL |
| | NO: 10 | FESGDQKDEAEKAKRMKEWMKRIKTTASEDEQEEMANAIITILQSWIFS |
| | SEQ ID | PKKKIQLHAEHALYDALMILNIXXXXXXXXXXXLEDYAFNFELILEEIACL |
| | NO: 11 | FESGXXKDEAEKAKRMKEWMKRIKTXXXEDEQEEMANAIITILQSWIFS |
| E53C | SEQ ID | PKKKIQLHAEHALYDALMILNIVKTNSPPAEEKLEDYAFNFELILEEIARL |
| | NO: 12 | FCSGDQKDEAEKAKRMKEWMKRIKTTASEDEQEEMANAIITILQSWIFS |
| | SEQ ID | PKKKIQLHAEHALYDALMILNIXXXXXXXXXXXLEDYAFNFELILEEIARL |
| | NO: 13 | FCSGXXKDEAEKAKRMKEWMKRIKTXXXEDEQEEMANAIITILQSWIFS |
| D56C | SEQ ID | PKKKIQLHAEHALYDALMILNIVKTNSPPAEEKLEDYAFNFELILEEIARL |
| | NO: 14 | FESGCQKDEAEKAKRMKEWMKRIKTTASEDEQEEMANAIITILQSWIFS |
| | SEQ ID | PKKKIQLHAEHALYDALMILNIXXXXXXXXXXXLEDYAFNFELILEEIARL |
| | NO: 15 | FESGCQKDEAEKAKRMKEWMKRIKTXXXEDEQEEMANAIITILQSWIFS |
| K58C | SEQ ID | PKKKIQLHAEHALYDALMILNIVKTNSPPAEEKLEDYAFNFELILEEIARL |
| | NO: 16 | FESGDQCDEAEKAKRMKEWMKRIKTTASEDEQEEMANAIITILQSWIFS |
| | SEQ ID | PKKKIQLHAEHALYDALMILNIXXXXXXXXXXXLEDYAFNFELILEEIARL |
| | NO: 17 | FESGXXCDEAEKAKRMKEWMKRIKTXXXEDEQEEMANAIITILQSWIFS |
| D59C | SEQ ID | PKKKIQLHAEHALYDALMILNIVKTNSPPAEEKLEDYAFNFELILEEIARL |
| | NO: 18 | FESGDQKCEAEKAKRMKEWMKRIKTTASEDEQEEMANAIITILQSWIFS |
| | SEQ ID | PKKKIQLHAEHALYDALMILNIXXXXXXXXXXXLEDYAFNFELILEEIARL |
| | NO: 19 | FESGXXKCEAEKAKRMKEWMKRIKTXXXEDEQEEMANAIITILQSWIFS |
| E62C | SEQ ID | PKKKIQLHAEHALYDALMILNIVKTNSPPAEEKLEDYAFNFELILEEIARL |
| | NO: 20 | FESGDQKDEACKAKRMKEWMKRIKTTASEDEQEEMANAIITILQSWIFS |
| | SEQ ID | PKKKIQLHAEHALYDALMILNIXXXXXXXXXXXLEDYAFNFELILEEIARL |
| | NO: 21 | FESGXXKDEACKAKRMKEWMKRIKTXXXEDEQEEMANAIITILQSWIFS |
| R66C | SEQ ID | PKKKIQLHAEHALYDALMILNIVKTNSPPAEEKLEDYAFNFELILEEIARL |
| | NO: 22 | FESGDQKDEAEKAKCMKEWMKRIKTTASEDEQEEMANAIITILQSWIFS |
| | SEQ ID | PKKKIQLHAEHALYDALMILNIXXXXXXXXXXXLEDYAFNFELILEEIARL |
| | NO: 23 | FESGXXKDEAEKAKCMKEWMKRIKTXXXEDEQEEMANAIITILQSWIFS |
| E69C | SEQ ID | PKKKIQLHAEHALYDALMILNIVKTNSPPAEEKLEDYAFNFELILEEIARL |
| | NO: 24 | FESGDQKDEAEKAKRMKCWMKRIKTTASEDEQEEMANAIITILQSWIFS |
| | SEQ ID | PKKKIQLHAEHALYDALMILNIXXXXXXXXXXXLEDYAFNFELILEEIARL |
| | NO: 25 | FESGXXKDEAEKAKRMKCWMKRIKTXXXEDEQEEMANAIITILQSWIFS |
| R73C | SEQ ID | PKKKIQLHAEHALYDALMILNIVKTNSPPAEEKLEDYAFNFELILEEIARL |
| | NO: 26 | FESGDQKDEAEKAKRMKEWMKCIKTTASEDEQEEMANAIITILQSWIFS |
| | SEQ ID | PKKKIQLHAEHALYDALMILNIXXXXXXXXXXXLEDYAFNFELILEEIARL |
| | NO: 27 | FESGXXKDEAEKAKRMKEWMKCIKTXXXEDEQEEMANAIITILQSWIFS |
| T77C | SEQ ID | PKKKIQLHAEHALYDALMILNIVKTNSPPAEEKLEDYAFNFELILEEIARL |
| | NO: 28 | FESGDQKDEAEKAKRMKEWMKRIKTCASEDEQEEMANAIITILQSWIFS |
| | SEQ ID | PKKKIQLHAEHALYDALMILNIXXXXXXXXXXXLEDYAFNFELILEEIARL |
| | NO: 29 | FESGXXKDEAEKAKRMKEWMKRIKTCASEDEQEEMANAIITILQSWIFS |
| E82C | SEQ ID | PKKKIQLHAEHALYDALMILNIVKTNSPPAEEKLEDYAFNFELILEEIARL |
| | NO: 30 | FESGDQKDEAEKAKRMKEWMKRIKTTASEDCQEEMANAIITILQSWIFS |
| | SEQ ID | PKKKIQLHAEHALYDALMILNIXXXXXXXXXXXLEDYAFNFELILEEIARL |
| | NO: 31 | FESGXXKDEAEKAKRMKEWMKRIKTXXXEDCQEEMANAIITILQSWIFS |
| E85C | SEQ ID | PKKKIQLHAEHALYDALMILNIVKTNSPPAEEKLEDYAFNFELILEEIARL |
| | NO: 32 | FESGDQKDEAEKAKRMKEWMKRIKTTASEDEQECMANAIITILQSWIFS |
| | SEQ ID | PKKKIQLHAEHALYDALMILNIXXXXXXXXXXXLEDYAFNFELILEEIARL |
| | NO: 33 | FESGXXKDEAEKAKRMKEWMKRIKTXXXEDEQECMANAIITILQSWIFS |

IL-2 receptor agonists of the present invention include IL-2 mimetics comprising a polypeptide at least 70% identical along its length to an amino acid sequence set forth in any one of SEQ ID NOs. 10-33. IL-2 receptor agonists of the present invention include IL-2 mimetics comprising a polypeptide at least 75% identical along its length to an amino acid sequence set forth in any one of SEQ ID NOs. 10-33. IL-2 receptor agonists of the present invention include IL-2 mimetics comprising a polypeptide at least 80% identical along its length to an amino acid sequence set forth in any one of SEQ ID NOs. 10-33. IL-2 receptor agonists of the present invention include IL-2 mimetics comprising a polypeptide at least 85% identical along its length to an amino acid sequence set forth in any one of SEQ ID NOs. 10-33. IL-2 receptor agonists of the present invention include IL-2 mimetics comprising a polypeptide at least 90%, identical along its length to an amino acid sequence set forth in any one of SEQ ID NOs. 10-33. IL-2 receptor agonists of the present invention include IL-2 mimetics comprising a polypeptide at least 95% identical along its length to an amino acid sequence set forth in any one of SEQ ID NOs. 10-33. IL-2 receptor agonists of the present invention include IL-2 mimetics comprising an amino acid sequence set forth in any one of SEQ ID NOs. 10-33. In illustrative such embodiments, the mutated cysteine is present (i.e. D56C; K58C; D59C; R66C; T77C; E85C; R50C; E53C; E62C; E69C; R73C; and/or E82C) and is optionally attached to a stability moiety such as, for example, a water stabilizing moiety such as a PEG-containing moiety, as set forth herein. In any of these embodiments, the polypeptide may be an IL-2 mimetic, wherein 1, 2, 3, 4, 5, 6, 7, 8, 9, 10, 11, 12, 13, or all 14 of the following are not true: position 7 is I, position 8 is T or M, position 11 is E, position 14 is K, position 18 is S, position 33 is Q, position 36 is R, position 37 is F, position 39 is K, position 40 is R, position 43 is R, position 44 is N, position 46 is W, and position 47 is G (numbering is in reference to SEQ ID NO:8. In a further embodiment, one or both of the following are not true: position 68 is I and position 98 is F (numbering is in reference to SEQ ID NO:8.

Typically for the IL-2 mimetics, one or more of the following are true: (i) X1 binds to the beta and the gamma subunit of the human IL-2 receptor; (ii) X2 does not bind to the human IL-2 receptor; (iii) X3 binds to the beta subunit of the human IL-2 receptor; (iv) X4 binds to the gamma subunit of the human IL-2 receptor; (v) the IL-2 mimetic does not bind to the alpha subunit of the human or murine IL-2 receptor. Binding to the receptors can be, for example, specific binding as determined by surface plasmon resonance at biologically relevant concentrations. In some aspects, the IL-2 mimetic polypeptides of any embodiment or combination of embodiments disclosed herein that bind to the IL-2 receptor β [symbol] _{c} heterodimer (IL-2Rβ [symbol] _{c}) do so with a binding affinity of 200 nm or less, 100 nm or less, 50 nM or less, or 25 nM or less.

The polypeptides and peptide domains described herein may include additional residues at the N-terminus, C-terminus, or both; these additional residues are not included in determining the percent identity of the polypeptides or peptide domains of the disclosure relative to the reference polypeptide. Such residues may be any residues suitable for an intended use, including but not limited to detection tags (i.e.: fluorescent proteins, antibody epitope tags, etc.), adaptors, ligands suitable for purposes of purification (His tags, etc.), other peptide domains that add functionality to the polypeptides, etc. Residues suitable for attachment of such groups may include cysteine, lysine or p-acetylphenylalanine residues or can be tags, such as amino acid tags suitable for reaction with transglutaminases as disclosed in U.S. Pat. Nos. 9,676,871 and 9,777,070.

IL-2 receptor agonists of the present invention include IL-2 mimetics having no disulfide bonds or at least one disulfide bond (i.e.: 1, 2, 3, 4, or more disulfide bonds). Any suitable disulfide bonds may be used, such as disulfide bonds linking two different helices. In one embodiment, the disulfide bonds include a disulfide bond linking helix 1 (X1) and helix 4 (X4). The disulfide bond may, for example, improve the thermal stability of the polypeptide as compared to a substantially similar polypeptide with no disulfide bond linking two domains together. In some aspects, the additional residues may be added to allow for disulfide bonds. For example, in one aspect, the N- and C-terminus of neoleukin-2/15 was remodeled to allow the introduction of a single-disulfide staple that encompasses the entire protein (added sequences CNSN (SEQ ID NO:27) and NFQC (SEQ ID NO:28), for N- and C-termini, respectively after removing terminal P and S residues.

### Conjugates and Fusions

The IL-2 receptor agonists of the present invention, including native IL-2 polypeptides, IL-2 active variants, and IL-2 mimetics, may be linked to other compounds to promote an increased half-life in vivo. Any such compounds can be used in the present invention provided that they are sufficiently safe to administer to a human subject. Examples include albumin, PEGylation (attachment of one or more polyethylene glycol chains), HESylation, PASylation, glycosylation, Fc-fusions or deimmunized variants. Such linkage can be covalent or non-covalent. In that regard, IL-2 variants have been developed that have amino acid substitutions that enable chemical conjugation with water soluble polymers (e.g., PEG) that increase circulating half-life compared to the IL-2 polypeptide alone. A “PEG” is a poly(ethylene glycol) molecule which is a water-soluble polymer of ethylene glycol. PEGs can be obtained in different sizes, and can also be obtained commercially in chemically activated forms that are derivatized with chemically reactive groups to enable covalent conjugation to proteins. Linear PEGs are produced in various molecular weights, such as PEG polymers of weight-average molecular weights of 5,000 daltons, 10,000 daltons, 20,000 daltons, 30,000 daltons, and 40,000 daltons. Branched PEG polymers have also been developed. Commonly-used activated PEG polymers are those derivatized with N-hydroxysuccinimide groups (for conjugation to primary amines such as lysine residues and protein N-termini), with aldehyde groups (for conjugation to N-termini), and with maleimide or iodoacetamide groups (for coupling to thiols such as cysteine residues). Methods of designing IL-2 moieties for conjugation to PEG are known in the art. For example, addition of polyethylene glycol (“PEG”) containing moieties may comprise attachment of a PEG group linked to maleimide group (e.g., “PEG-MAL”) to a cysteine residue of the polypeptide. Suitable examples of PEG-MAL include, but are not limited to, methoxy PEG-MAL 5 kD; methoxy PEG-MAL 20 kD; methoxy (PEG)2-MAL 40 kD; methoxy PEG(MAL)2 5 kD; methoxy PEG(MAL)2 20 kD; methoxy PEG(MAL)2 40 kD; or any combination thereof. See also U.S. Pat. No. 8,148,109. The skilled artisan will be able to use the methods described herein or alternative methods to design long-acting IL-2 receptor agonists for use in the present invention.

Exemplary PEGylated IL-2 receptor agonists of the present invention include IL-2 mimetics comprising the amino acid sequence set forth in SEQ ID NO:20 (NEO 2-15 E62C), wherein the cysteine at position 62 is PEGlyated. The polyethylene group can be attached via any suitable attachment chemistry, including, for example, with maleimide (e.g., maleimide-modified PEG (PEG-MAL) 5 kD; PEG-MAL 20 kD; or PEG-MAL 40 kD). In some embodiments, the PEGylation is with PEG-MAL 30 kD. In some embodiments, the PEGylation is with modified PEG-MAL 40 kD. In some embodiments, the range for repeating PEG units in the PEGylated peptide of SEQ ID NO:20 is about 800-1000. In some embodiments, the average number of repeating PEG units in the PEGylated peptide of SEQ ID NO:20 is about 850-950. One of skill in the art will understand that PEG portions can be linear or branched.

Exemplary PEGylated IL-2 receptor agonists of the present invention include IL-2 mimetics comprising the amino acid sequence set forth in SEQ ID NO:30 (NEO 2-15 E82C), wherein the cysteine at position 82 is PEGlyated. The polyethylene group can be attached via any suitable attachment chemistry, including, for example, with maleimide-modified PEG (PEG-MAL) 5 kD; PEG-MAL 20 kD; or PEG-MAL 40 kD.) In some embodiments, the PEGylation is with PEG-MAL 30 kD. In some embodiments, the PEGylation is with PEG-MAL 40 kD. In some embodiments, the range for repeating PEG units in the PEGylated peptide of SEQ ID NO:30 is about 800-1000. In some embodiments, the average number of repeating PEG units in the PEGylated peptide of SEQ ID NO:30 is about 850-950. One of skill in the art will understand that PEG portions can be linear or branched.

Exemplary PEGylated IL-2 receptor agonists of the present invention include IL-2 mimetics comprising the amino acid sequence set forth in SEQ ID NO:24 (NEO 2-15 E69C), wherein the cysteine at position 69 is PEGlyated. The polyethylene group can be attached via any suitable attachment chemistry, including, for example, with maleimide-modified PEG (e.g., PEG-MAL 5 kD; PEG-MAL 20 kD; or PEG-MAL 40 kD). In some embodiments, the PEGylation is with PEG-MAL 30 kD. In some embodiments, the PEGylation is with modified PEG-MAL 40 kD. In some embodiments, the range for repeating PEG units in the PEGylated peptide of SEQ ID NO:24 is about 800-1000. In some embodiments, the average number of repeating PEG units in the PEGylated peptide of SEQ ID NO:24 is about 850-950. One of skill in the art will understand that PEG portions can be linear or branched.

Exemplary PEGylated IL-2 receptor agonists of the present invention include IL-2 mimetics comprising the amino acid sequence set forth in SEQ ID NO:26 (NEO 2-15 R73C), wherein the cysteine at position 73 is PEGlyated. The polyethylene group can be attached via any suitable attachment chemistry, including, for example, with maleimide-modified PEG (PEG-MAL) 5 kD; PEG-MAL 20 kD; or PEG-MAL 40 kD.) In some embodiments, the PEGylation is with PEG-MAL 30 kD. In some embodiments, the PEGylation is with modified PEG-MAL 40 kD. In some embodiments, the range for repeating PEG units in the PEGylated peptide of SEQ ID NO:26 is about 800-1000. In some embodiments, the average number of repeating PEG units in the PEGylated peptide of SEQ ID NO:26 is about 850-950. One of skill in the art will understand that PEG portions can be linear or branched.

The IL-2 receptor agonists of the present invention include IL-2 receptor agonist fusion proteins and IL-2 receptor agonist conjugates, including for example, IL-2-receptor agonist-Fc fusion protein, IL-2-receptor agonist-CD25 fusion proteins, IL-2-receptor agonist-targeting domain fusion proteins or IL-2-receptor agonist-targeting domain conjugates. The targeting domains are polypeptide domains or small molecules that bind to a target of interest. The targeting domain may be covalently or non-covalently bound to the polypeptide. In embodiments where the targeting domain is non-covalently bound to the polypeptide, any suitable means for such non-covalent binding may be used, including but not limited to streptavidin-biotin linkers. In another embodiment, the targeting domain, when present, is a translational fusion with the IL-receptor agonist. In this embodiment, the polypeptide and the targeting domain may directly abut each other in the translational fusion or may be linked by a polypeptide linker suitable for an intended purpose. Exemplary such linkers include, but are not limited, to those disclosed in WO2016178905, WO2018153865, and WO 2018170179.

The targeting domain can bind, for example, to a cell surface protein or an immune cell surface marker. With respect to a cell surface protein, the cell can be any cell type of interest that includes a surface protein that can be bound by a suitable targeting domain. In one embodiment, the cell surface proteins are present on the surface of cells selected from the group consisting of tumor cells, tumor vascular component cells, tumor microenvironment cells (e.g. fibroblasts, infiltrating immune cells, or stromal elements), and other cancer cells and immune cells (including but not limited to CD8+ T cells, T-regulatory cells, dendritic cells, NK cells, or macrophages). When the cell surface protein is on the surface of a tumor cell, vascular component cell, or tumor microenvironment cell (e.g. fibroblasts, infiltrating immune cells, or stromal elements), any suitable tumor cell, vascular component cell, or tumor microenvironment cell surface marker may be targeted.

In another embodiment, the targeting domain can bind to, for example, immune cell surface markers. In this embodiment, the target may be cell surface proteins on any suitable immune cell, including but not limited to CD8+ T cells, T-regulatory cells, dendritic cells, NK cells or macrophages. The targeting domain may target any suitable immune cell surface marker.

When a targeting domain is a polypeptide, the targeting domain can be any suitable polypeptide that bind to one or more targets of interest and can be attached or associated with an IL-2 polypeptide, including IL-2 mimetic. In non-limiting embodiments, the targeting domain may include but is not limited to an scFv, a F(ab), a F(ab′)₂, a B cell receptor (BCR), a DARPin, an affibody, a monobody, a nanobody, diabody, an antibody (including a monospecific or bispecific antibody); a cell-targeting oligopeptide including but not limited to RGD integrin-binding peptides, de novo designed binders, aptamers, a bicycle peptide, conotoxins, small molecules such as folic acid, and a virus that binds to the cell surface. Methods of making IL-2 fusion proteins and conjugates are known in the art and not discussed herein in detail.

In some aspects, an IL-2 receptor agonist of the present invention is fused or conjugated to a moiety, such as a targeting agent. In some aspects, the targeting agent is an antibody. In other aspects, the targeting agent is a moiety other than an antibody. In some embodiments, the IL-2 receptor agonists are not targeted. In some aspects, an IL-2 receptor agonist of the present invention is a fusion protein. In other aspects, an IL-2 receptor agonist of the present invention is not a fusion protein. In some aspects, the IL-2 receptor agonists of the present invention are conjugated to a targeting agent but the targeting agent is not directed against the fibroblast activation protein (FAP).

### Disease States

The present disclosure provides, inter alia, methods for modulating an immune response in a subject by administering to the subject an IL-2 receptor agonist of the present invention.

As used herein, an “immune response” being modulated refers to a response by a cell of the immune system, such as a B cell, T cell (CD4 or CD8), regulatory T cell, antigen-presenting cell, dendritic cell, monocyte, macrophage, NKT cell, NK cell, basophil, eosinophil, or neutrophil, to a stimulus. In some embodiments, the response is specific for a particular antigen (an “antigen-specific response”) and refers to a response by a CD4 T cell, CD8 T cell, or B cell via their antigen-specific receptor. In some embodiments, an immune response is a T cell response, such as a CD4+ response or a CD8+ response. Such responses by these cells can include, for example, cytotoxicity, proliferation, cytokine or chemokine production, trafficking, or phagocytosis, and can be dependent on the nature of the immune cell undergoing the response. In some embodiments of the compositions and methods described herein, an immune response being modulated is T-cell mediated. Methods of measuring an immune response are known in the art and include, for example, measuring pro-inflammatory cytokines such as IL-6, IL-12 and TNF-alpha as well as co-stimulatory molecules, such as CD80, CD86, and chemokine receptor.

In a further aspect, the present disclosure provides methods for treating cancer, comprising administering to a subject in need thereof a dosage regimen as described herein. As used herein, “treat” or “treating” means accomplishing one or more of the following: (a) reducing the size or volume of tumors and/or metastases in the subject; (b) limiting any increase in the size or volume of tumors and/or metastases in the subject; (c) increasing survival; (d) reducing the severity of symptoms associated with cancer; (e) limiting or preventing development of symptoms associated with cancer; and (f) inhibiting worsening of symptoms associated with cancer. The term therapeutically effective amount or therapeutically effective dose level is an amount or dose level sufficient to show benefit to a patient (e.g., by treating a patient).

The methods can be used to treat cancer, including but not limited to colon cancer, melanoma, renal cell cancer, head and neck squamous cell cancer, gastric cancer, urothelial carcinoma, Hodgkin lymphoma, non-small cell lung cancer, small cell lung cancer, hepatocellular carcinoma, pancreatic cancer, Merkel cell carcinoma, colorectal cancer, acute myeloid leukemia, acute lymphoblastic leukemia, chronic lymphocytic leukemia, sarcoma, non-Hodgkin lymphoma, multiple myeloma, ovarian cancer, cervical cancer, breast cancer, liver cancer, renal cell carcinoma, melanoma, and any tumor types selected by a diagnostic test, such as microsatellite instability, tumor mutational burden, PD-L1 expression level, or the immunoscore assay (as developed by the Society for Immunotherapy of Cancer). In some aspects, the cancer to be treated is a solid tumor cancer. In other embodiments, the cancer is a hematologic malignancy.

The IL-2 receptor agonists may be administered together with (i.e.: combined or separately) one or more other prophylactic or therapeutic agents, including but not limited to tumor resection, chemotherapy, radiation therapy, immunotherapy, and the like.

### Pharmaceutical Compositions

Pharmaceutical compositions can be formulated so as to allow the IL-2 receptor agonists to be bioavailable upon administration of the composition to a patient. The IL-2 receptor agonists can take the form of solutions, suspensions, emulsion, microparticles, tablets, pills, pellets, capsules, capsules containing liquids, powders, sustained-release formulations, suppositories, emulsions, aerosols, sprays, suspensions, or any other form suitable for use. Other examples of suitable pharmaceutical carriers are described in “Remington's Pharmaceutical Sciences” by E. W. Martin. It will be evident to those of ordinary skill in the art that the optimal dosage of the active ingredient(s) in the pharmaceutical composition will depend on a variety of factors. Relevant factors include, without limitation, the type of animal (e.g., human), the particular form of the IL-2 receptor agonists, the manner of administration, and the composition employed.

The pharmaceutically acceptable carrier or vehicle can be particulate, so that the compositions are, for example, in tablet or powder form. The carrier(s) can be liquid, with the compositions being, for example, an oral syrup or injectable liquid. In addition, the carrier(s) can be gaseous or particulate, so as to provide an aerosol composition useful in, e.g., inhalatory administration.

When intended for oral administration, the IL-2 receptor agonist is preferably in solid or liquid form, where semi-solid, semi-liquid, suspension and gel forms are included within the forms considered herein as either solid or liquid. As a solid composition for oral administration, the composition can be formulated into a powder, granule, compressed tablet, pill, capsule, chewing gum, wafer or the like form. Such a solid composition typically contains one or more inert diluents. In addition, one or more of the following can be present: binders such as carboxymethylcellulose, ethyl cellulose, microcrystalline cellulose, or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin, a flavoring agent such as peppermint, methyl salicylate or orange flavoring, and a coloring agent.

When the composition is in the form of a capsule, e.g., a gelatin capsule, it can contain, in addition to materials of the above type, a liquid carrier such as polyethylene glycol, cyclodextrin or a fatty oil. The composition can be in the form of a liquid, e.g., an elixir, syrup, solution, emulsion or suspension. The liquid can be useful for oral administration or for delivery by injection. When intended for oral administration, a composition can comprise one or more of a sweetening agent, preservatives, dye/colorant and flavor enhancer. In a composition for administration by injection, one or more of a surfactant, preservative, wetting agent, dispersing agent, suspending agent, buffer, stabilizer and isotonic agent can also be included. Also contemplated are delayed release capsule, including those with an enteric coating.

The liquid compositions, whether they are solutions, suspensions or other like form, can also include one or more of the following: sterile diluents such as water for injection, saline solution, preferably physiological saline, Ringer's solution, isotonic sodium chloride, fixed oils such as synthetic mono or digylcerides which can serve as the solvent or suspending medium, polyethylene glycols, glycerin, cyclodextrin, propylene glycol or other solvents; antibacterial agents such as benzyl alcohol or methyl paraben; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. A parenteral composition can be enclosed in ampoule, a disposable syringe or a multiple-dose vial made of glass, plastic or other material. Physiological saline is an exemplary adjuvant. An injectable composition is preferably sterile.

The present disclosure provides pharmaceutical compositions, comprising one or more agonists of the disclosure and a pharmaceutically acceptable carrier. The term “carrier” refers to a diluent, adjuvant or excipient, with which an IL2 receptor agonist is administered. The pharmaceutical composition may comprise, for example, in addition to the IL-2 receptor agonist (a) a lyoprotectant; (b) a surfactant; (c) a bulking agent; (d) a tonicity adjusting agent; (e) a stabilizer; (f) a preservative and/or (g) a buffer.

In some embodiments, the buffer in the pharmaceutical composition is a Tris buffer, a histidine buffer, a phosphate buffer, a citrate buffer or an acetate buffer. The pharmaceutical composition may also include a lyoprotectant, e.g. sucrose, sorbitol or trehalose. In certain embodiments, the pharmaceutical composition includes a preservative e.g. benzalkonium chloride, benzethonium, chlorohexidine, phenol, m-cresol, benzyl alcohol, methylparaben, propylparaben, chlorobutanol, o-cresol, p-cresol, chlorocresol, phenylmercuric nitrate, thimerosal, benzoic acid, and various mixtures thereof. In other embodiments, the pharmaceutical composition includes a bulking agent, like glycine. In yet other embodiments, the pharmaceutical composition includes a surfactant e.g., polysorbate-20, polysorbate-40, polysorbate-60, polysorbate-65, polysorbate-80 polysorbate-85, poloxamer-188, sorbitan monolaurate, sorbitan monopalmitate, sorbitan monostearate, sorbitan monooleate, sorbitan trilaurate, sorbitan tristearate, sorbitan trioleaste, or a combination thereof. The pharmaceutical composition may also include a tonicity adjusting agent, e.g., a compound that renders the formulation substantially isotonic or isoosmotic with human blood. Exemplary tonicity adjusting agents include sucrose, sorbitol, glycine, methionine, mannitol, dextrose, inositol, sodium chloride, arginine and arginine hydrochloride. In other embodiments, the pharmaceutical composition additionally includes a stabilizer, e.g., a molecule which, when combined with a protein of interest substantially prevents or reduces chemical and/or physical instability of the protein of interest in lyophilized or liquid form. Exemplary stabilizers include sucrose, sorbitol, glycine, inositol, sodium chloride, methionine, arginine, and arginine hydrochloride.

The IL-2 receptor agonists may be the sole active agent in the pharmaceutical composition, or the composition may further comprise one or more other active agents suitable for an intended use.

### Treatment Regimen

The Il-2 receptor agonists can be administered by any convenient route, for example by infusion or bolus injection, by absorption through epithelial or mucocutaneous linings (e.g., oral mucosa, rectal and intestinal mucosa, etc.). Administration can be systemic or local. Typical routes of administration include, without limitation, oral, topical, parenteral, sublingual, rectal, vaginal, ocular, intra-tumor, and intranasal. Parenteral administration includes subcutaneous injections, intravenous, intramuscular, intrasternal injection or infusion techniques. In one aspect, the IL-2 receptor agonists are administered parenterally. In yet another aspect, the IL-2 receptor agonists are administered intravenously or subcutaneously. In specific embodiments, it can be desirable to administer an IL-2 receptor agonist locally to the area in need of treatment. In one embodiment, administration can be by direct injection at the site (or former site) of a cancer, tumor or neoplastic or pre-neoplastic tissue. In another embodiment, administration can be by direct injection at the site (or former site) of a manifestation of an autoimmune disease. An example of local administration is infusion via a catheter, e.g., intravesical infusion.

The methods of the present invention provide for the administration of one or more priming doses of an IL-2 receptor agonist as well as administration of the IL-2 receptor agonist at a target dose level. Prior to administration of the priming dose and the target dose to a subject, the priming dose and target dose to be administered to the subject will be selected. As noted herein, the priming dose is reduced as compared to the target dose or in other words, the target dose is always greater than the priming dose.

It is important to note that the term priming dose or priming dose level, as used herein, refers to each individual priming dose and not to the sum of the individual priming doses. For example, if two priming doses of 0.5 ug/kg are administered to a subject, the priming dose or the priming dose level is 0.5 ug/kg for the first priming dose and 0.5 ug/kg for the second priming dose and not 1 ug/kg. Similarly, if a priming dose of 0.5 ug/kg and 1 ug/kg are administered to a subject, the priming dose or priming dose level is 0.5 ug/kg for the first priming dose and 1 ug/kg for the second priming dose, and not 1.5 ug/kg. In some aspects, the target dose level and the priming dose level to be selected and administered will be therapeutically effective dose levels. In some aspects, the target dose level will be at the maximum tolerated dose (MTD). In some aspects, for maximum therapeutic effect, the priming dose and the target dose will be provided at the MTD, keeping in mind that the MTD will be different for an initiation dose and for a later delivered dose. In some aspects, one priming dose will be administered to a subject. In other aspects, one to six, one to five, one to four, one to three, or two priming doses will be administered to a subject.

In some aspects, the target dose level is at least 10%, at least 20%, at least 25% at least 30%, at least 40%, at least 50%, at least 60%, at least 70%, or at least 75% greater than the initial priming dose level. In some aspects, the target dose level is double or more than double (e.g., triple) the initial priming dose level. In some aspects, the target dose level is at least 4 to 6 times greater than the initial priming dose level. In some aspects, the target dose level is no more than double or no more than triple than the initial priming dose level. In other aspects, the target dose is level at least 10%, at least 20%, at least 25%, at least 30%, at least 40%, at least 50%, at least 60%, at least 70%, or at least 75% greater than each of the priming dose levels. In some aspects, the target dose level is double or more than double each of the priming dose levels. In some aspects, the target dose level is no more than double or no more than triple than each of the priming dose levels.

The present methods contemplate embodiments wherein a subject will receive only one priming dose prior to administration at a target dose level. In other aspects, a subject will receive two or more priming doses prior to administration at a target dose level. In some aspects, two or more priming doses are administered over a period of one to ten, one to five or one to three days. In some aspects, two or more priming doses are administered with at least 7 days between each dose. In some aspects, two or more priming doses are administered with at least 20 days between each dose. The priming dose levels can be substantially the same or they can be varied. For example, in some aspects, two or more priming doses will be administered at escalating dose levels. In other aspects, the initial priming dose will be at a higher dose level than the subsequent priming doses. Following the first administration of the IL-2 receptor agonist at the target dose level, subsequent doses can be administered at the target dose level. In some aspects, at least two to eight subsequent doses will be administered at the target dose level at intervals of between about 7 to about 21 days. In some aspects, following administration of drug at the target dose level, it may be desirable to put the subject on a maintenance therapy wherein the maintenance therapy dose is at a decreased dosage level as compared to the target level. In some aspects, following administration of drug at the target dose level, it may be desirable to put the subject on a maintenance therapy wherein the maintenance therapy dose is at the same dosage level or decreased dosage level as compared to the priming dose level. In some aspects, prior to administration of the target dose in the subject, sufficient time is allowed for an increase in lymphocyte production in the subject.

The amount of the IL-2 receptor agonist that is effective in the treatment of a particular disorder or condition will depend on the nature of the disorder or condition, and can be determined by standard clinical techniques, in combination with the teachings of the present application. In addition, in vitro or in vivo assays can optionally be employed to help identify optimal dosage ranges. The precise dose to be employed will also depend on the route of administration, and the seriousness of the disease or disorder, and should be decided according to the judgment of the practitioner and each patient's circumstances, using the teachings described herein, in particular the teachings that by administering to the subject one or more priming doses of the IL-2 receptor agonist, a dose level that would have an unacceptable tolerability profile if administered to the subject as a first dose, can be subsequently administered. In some aspects, the priming dose is from 0.01 ug/kg to 1 mg/kg, 0.1 ug/kg to 10 ug/kg, 0.1 ug/kg to 5 ug/kg, 0.1 ug/kg to 2 ug/kg, 0.5 ug/kg to 20 ug/kg, 0.5 ug/kg to 10 ug/kg, or from 0.5 ug/kg to 3 ug/kg. In some aspects, the target dose level is from 0.1 ug/kg to 2 mg/kg, 0.2 ug/kg to 1 mg/kg, 0.2 ug/kg to 50 ug/kg, 0.2 ug/kg to 20 ug/kg, 0.2 ug/kg to 50 ug/kg, 0.2 ug/kg to 10 ug/kg, 0.2 ug/kg to 8 ug/kg, 1 ug/kg to 50 ug/kg, 1.5 ug/kg to 30 ug/kg, 2 ug/kg to 35 ug/kg, or from 4 ug/kg to 20 ug/kg. In some aspects, the priming dose is from 0.01 ug/kg to 1 mg/kg and the target dose level is from 0.1 ug/kg to 2 mg/kg. In some aspects, the priming dose is from 0.1 ug/kg to 10 ug/kg, from 0.1 ug/kg to 5 ug/kg, or from 0.1 ug/kg to 2 ug/kg and the target dose level is from 0.2 ug/kg to 1 mg/kg, from 0.2 ug/kg to 50 ug/kg, from 0.2 ug/kg to 20 ug/kg, and any combinations thereof provided that the target dose is always than the priming dose. In some aspects, the priming dose is from 0.1 ug/kg to 5 ug/kg, or from 0.1 ug/kg to 2 ug/kg and the target dose level is from 0.2 ug/kg to 1 mg/kg, from 0.2 ug/kg to 50 ug/kg, from 0.2 ug/kg to 20 ug/kg, from 0.2 ug/kg to 10 ug/kg, or from 0.2 ug/kg to 8 ug/kg and any combinations thereof provided that the target dose is greater than the priming dose. In other aspects, the priming dose is from 0.5 ug/kg to 20 ug/kg, from 0.5 ug/kg to 10 ug/kg, or from 0.5 ug/kg to 3 ug/kg and the target dose level is from 1 ug/kg to 50 ug/kg, or from 2 ug/kg to 35 ug/kg and any combinations thereof provided that the target dose is greater than the priming dose. In other aspects, the priming dose is from 0.5 ug/kg to 10 ug/kg, or from 0.5 ug/kg to 3 ug/kg and the target dose level is from 1 ug/kg to 50 ug/kg, from 2 ug/kg to 35 ug/kg or from 4 ug/kg to 20 ug/kg and any combinations thereof provided that the target dose is greater than the priming dose. In some aspects, the priming dose is from 0.25 ug/kg to 20 ug/kg and the target dose level is from 1.5 ug/kg to 30 ug/kg provided that the target dose is greater than the priming dose. In some aspects, the priming dose is selected from the group consisting of 0.25 ug/kg, 1.5 ug/kg, 3 ug/kg, 6 ug/kg, 12 ug/kg, and 20 ug/kg. In some aspects, the target dose level is selected from the group consisting of 1.5 ug/kg, 3 ug/kg, 6 ug/kg, 12 ug/kg, 20 ug/kg, and 30 ug/kg.

In some embodiments, the target dose is 1.5 ug/kg, and one or more priming dose(s) of 0.25 ug/kg is/are administered during a first dosing cycle before administering the 1.5 ug/kg target dose. In some embodiments, the target dose is 3 ug/kg, and priming doses of 0.25 ug/kg and 1.5 ug/kg are administered during the first two dosing cycles, respectively, before administering the 3 ug/kg target dose. In some embodiments, the target dose is 6 ug/kg, and priming doses of 0.25 ug/kg, 1.5 ug/kg, and 3 ug/kg are administered during the first three dosing cycles, respectively, before administering the 6 ug/kg target dose. In some embodiments, the target dose is 12 ug/kg, and priming doses of 0.25 ug/kg, 1.5 ug/kg, 3 ug/kg, and 6 ug/kg are administered during the first four dosing cycles, respectively, before administering the 20 ug/kg target dose. In some embodiments, the target dose is 20 ug/kg, and priming doses of 0.25 ug/kg, 1.5 ug/kg, 3 ug/kg, 6 ug/kg, and 12 ug/kg are administered during the first five dosing cycles, respectively before administering the 20 ug/kg target dose. In some embodiments, the target dose is 30 ug/kg, and priming doses of 0.25 ug/kg, 1.5 ug/kg, 3 ug/kg, 6 ug/kg, 12 ug/kg, and 20 ug/kg are administered during the first six dosing cycles, respectively, before administering the 30 ug/kg target dose. In some embodiments, only one priming dose is administered during a dosing cycle. In some embodiments, two priming doses are administered during a dosing cycle. In some embodiments, the priming and target doses are administered on a 21-day cycle. For example, in some such embodiments, a priming dose will be administered on day 1 of a first 21 day cycle and the target dose will be administered on day 1 of a second 21 day cycle. In some embodiments, the priming and target doses are administered on days 1 and 8 of a 21-day cycle. For example, in some such embodiments, a priming dose will be administered on day 1 and 8 of a first 21 day cycle, and the target dose will be administered on day 1 and day 8 of a second 21 day cycle. In particularly preferred embodiments, the IL-2 receptor agonist is a PEGylated IL-2 mimetic as described herein. For all of the values herein for target doses and priming doses, it will be understood by the skilled practitioner that each priming dose is always a reduced dose as compared to the target dose. For example, in embodiments wherein the priming dose is selected from the group consisting of 0.25 ug/kg, 1.5 ug/kg, 3 ug/kg, 6 ug/kg, 12 ug/kg, and 20 ug/kg and the target dose is selected from the group consisting of 1.5 ug/kg, 3 ug/kg, 6 ug/kg, 12 ug/kg, 20 ug/kg, and 30 ug/kg, if the target dose is 1.5 ug/kg, the priming dose will be 0.25 ug/kg. The priming dose can be administered one or more times. Similarly, if the target dose is 20 ug/kg, the priming dose can be selected from 0.25 ug/kg, 1.5 ug/kg, 3 ug/kg, 6 ug/kg, or 12 ug/kg. The priming dose can be administered one or more times. For example, a priming dose of 12 ug/kg can be administered two times prior to the target dose of 20 ug/kg.

In some aspects, the target dose level and the priming dose level will be therapeutically effective dose levels. In some aspects, the target dose level will be at the maximum tolerated dose (MTD). In some such embodiments, at least one of the priming doses will be provided at the maximum tolerated dose, keeping in mind that the MTD will be different for an initiation dose and for a later delivered dose. In other embodiments, the priming doses will be below the MTDs.

Exemplary IL-2 receptor agonists include, for example, bempegaldesleukin, MDNA109, THOR-707, R06874281 and the IL-2 mimetics described herein (including conjugated, fused and targeted versions thereof). Bempegaldesleukin (also referred to as NKTR-214) is a human recombinant IL-2 attached to average of 6 releasable polyethylene glycol chains and has the generic name of bempegaldesleukin. THOR-707 is a variant of recombinant human IL-2 that is pegylated at one specific site, designed to block engagement of the IL-2 receptor alpha chain. ALKS-4230 (Alkermes) is an engineered fusion protein comprising a circularly permuted IL-2 and the extracellular portion of the IL-2 receptor alpha. MDNA109 (Medicenna) is a version of IL-2 that includes several amino acid substitutions to increase binding affinity to IL-2RB.

Methods of dosing these IL-2 receptor agonists (e.g., bempegaldesleukin, ALKS-4230, MDNA109, IL-2 mimetics) as well as other IL-2 receptor agonists according to the present invention would include, for example, administering to the subject a priming dose of the IL-2 receptor agonist prior to administration of a higher target dose. The priming dose level can be, for example, 90%, 80%, 70%, 60%, 50%, 40%, 30%, 20% or 10% of the target dose. Expressed otherwise, the target dose level can be, for example, at least 10%, at least 20%, at least 30%, at least 40%, at least 50%, at least 60%, at least 70%, or at least 75% greater than the priming dose level.

The timing between administration of the first priming dose and administration of the IL-2 receptor agonist at a target dose level can vary. In some aspects, administration of the IL-2 receptor agonist at the target dose level is at least 5 days, at least 6 days, or at least 7 days following administration of the first priming dose. In other aspects, administration of the IL-2 receptor agonist at the target dose level is at least 7 or at least 8 days following administration of the first priming dose. In some aspects, the first administration of the IL-2 receptor agonist at the target dose level is on day 5, day 6, day 7, or day 8 following administration of the first priming dose. In some aspects, the first administration of the IL-2 receptor agonist at the target dose level is no more than 10 days, no more than 14 days, no more than 21 or 22 days, no more than 30 days, or no more than 63 days following administration of the first priming dose. In some aspects, the first administration of the IL-2 receptor agonist at the target dose level is no more than 10 days, no more than 14 days, no more than 21 or 22 days, no more than 30 days, or no more than 63 days following administration of the last priming dose. In some aspects, the first administration of the IL-2 receptor agonist at the target dose level is at any one of days 5 to 21, 5 to 22, 5 to 14, 7 to 21, 7 to 22, 7 to 14, or 7 to 63 following administration of the first priming dose. In some aspects, the first administration of the IL-2 receptor agonist at the target dose level is at any one of days 5 to 21, 5 to 22, 5 to 14, 7 to 21, 7 to 22, 7 to 14, or 7 to 63 following administration of the last priming dose. In some embodiments wherein more than two doses of the IL-2 receptor agonist are administered to the subject following administration of the IL-2 receptor agonist at the target dose level, the timing between administration of the target doses can vary as well. For example, administration can be daily or weekly. Administration can be, for example, once every 6-12 hours, once every two weeks, once every three weeks, once every four weeks, once every five weeks, once every 6 weeks, once every 7 weeks, or once every 8 weeks. In one embodiment, administration is once every 3 weeks such as on day 1 of a 21-day cycle. In another embodiment, for example, administration is on day 1 and day 8 of a three week cycle. In some aspects, the doses are separated in time from each other by at least 7 days. In some aspects, the doses are separated in time from each other by at least 8 days. In some aspects, the doses are separated in time from each other by at least 21 days. In some aspects, the doses are separated in time from each other by no more than 8 days, no more than two weeks, no more than three weeks or no more than four weeks. In some aspects, following the first administration of the IL-2 receptor agonist at the target dose level, two to eight subsequent doses are administered at the target dose level at intervals of between 7 to 21 days.

Current dosing being investigated for bempegaldesleukin is 6 ug/kg IV every 3 weeks. One example of a dosing schedule according to the present invention would be 4 ug/kg IV on day 1, followed by 6 ug/kg IV every 2 weeks starting on day 15, or followed by 8 ug/kg every 3 weeks starting on day 22; or 2 ug/kg IV on day 1, followed by 4 ug/kg IV every 1 week starting on day 8. Current dosing for ALKS-4230 is 6 ug/kg IV daily for 5 doses. One example of a dosing schedule according to the present invention would be 4 ug/kg IV on day 1 and 8 ug/kg IV daily for 4 doses starting on day 2

In some aspects, the dosing regimens provided herein increase a subject's probability of responding to the therapy as compared to other dosing regimens. In some aspects, the dosing regimens provided herein decrease a subject's probability of suffering from an adverse event (including a dose limiting toxicity) as compared to other dosing regimens. An exemplary comparative dosing regimen is one that uses the same dosage level at initiation and throughout the first months of treatment. In such exemplary comparative dosing regimens, there is no priming dose and the target dose is administered at initiation of treatment. As used herein, “adverse event” refers to a harmful, deleterious and/or undesired effect of administering an IL-2 receptor agonist to a subject. Adverse events are graded on toxicity and various toxicity scales exist providing definitions for each grade. Exemplary of such scales are toxicity scales of the National Cancer Institute Common Toxicity Criteria version 2.0, the World Health Organization or Common Terminology Criteria for Adverse Events (CTCAE) scale. Generally, the scale is as follows: Grade 1=mild side effects; Grade 2=moderate side effects; Grade 3=Severe side effects; Grade 4=Life Threatening or Disabling side-effects; Grade 5=Fatal. Assigning grades of severity is within the experience of a physician or other health care professional.

A dose level that would have an unacceptable tolerability profile is one that would cause unacceptable side effects or overt toxicity in a specific period of time. Such toxicities, for example, are typically grade 3 and higher (e.g., grade 3 or 4) and may require hospital-based supportive treatment although they may include persistent grade 2 toxicities that fail to resolve over the course of treatment and that limits the patient's ability to comply with the protocol therapy. A clinician will be able to make a determination based on the totality of the data (including type and frequency of toxicities) as to whether a particular dose level is associated with an unacceptable tolerability profile.

In some aspects, a dose level that would have an unacceptable tolerability profile is one that is above the maximum tolerate dose (MTD). The present inventions have found that for the long-acting IL-2 receptor agonists, the MTD will be different for an initiation dose and for a later delivered dose. The maximum tolerated dose can be determined in clinical trials by testing increasing doses on different groups of people until the highest dose with acceptable side effects is found. It is within the level of skill of a skilled physician to assign or determine a MTD depending on the treatment protocol, the disease to be treated, the dosage regime and the particular patient to be treated. The teachings provided herein regarding administration of a priming dose prior to a target dose of an IL-2 receptor agonist can be used to improve the tolerability of the IL-2 receptor agonist.

In some particularly preferred embodiments, the IL-2 receptor agonist is a PEGylated IL-2 mimetic as described herein and the priming and target doses are as follows:
(i) the one or more priming doses are from 0.1 ug/kg to 10 ug/kg, from 0.1 ug/kg to 5 ug/kg, or from 0.1 ug/kg to 2 ug/kg and the target dose is from 0.2 ug/kg to 20 ug/kg, provided that the target dose is greater than the priming dose;
(ii) the one or more priming doses are from 0.1 ug/kg to 5 ug/kg or from 0.1 ug/kg to 2 ug/kg and the target dose is from 0.2 ug/kg to 20 ug/kg, from 0.2 ug/kg to 10 ug/kg, or from 0.2 ug/kg to 8 ug/kg, provided that the target dose is greater than the priming dose;
(iii) the one or more priming doses are from 0.5 ug/kg to 10 ug/kg, or from 0.5 ug/kg to 3 ug/kg and the target dose is from 2 ug/kg to 35 ug/kg, provided that the target dose is greater than the priming dose;
(iv) the one or more priming doses are from 0.5 ug/kg to 10 ug/kg or from 0.5 ug/kg to 3 ug/kg and the target dose is from 2 ug/kg to 35 ug/kg, provided that the target dose is greater than the priming dose;
(v) the one or more priming doses are from 0.5 ug/kg to 10 ug/kg or from 0.5 ug/kg to 3 ug/kg and the target dose is from 4 ug/kg to 20 ug/kg, provided that the target dose is greater than the priming dose;
(vi) the one or more priming doses are from 0.25 ug/kg to 20 ug/kg and the target dose is from 1.5 ug/kg to 30 ug/kg, provided that the target dose is greater than the priming dose; or
(vii) the one or more priming doses are from 0.25 ug/kg to 4.5 ug/kg and the target dose is from 1.5 ug/kg to 10 ug/kg, provided that the target dose is greater than the priming dose.

In some particularly preferred embodiments, the IL-2 receptor agonist is a PEGylated IL-2 mimetic as described herein and the priming and target doses are as follows:
(i) the target dose is 1 ug/kg and the one or more priming doses are 0.1 ug/kg, 0.15 ug/kg, 0.25 ug/kg, 0.3 ug/kg, or 0.5 ug/kg or a combination thereof;
(ii) the target dose is 3 ug/kg and the one or more priming doses are 1.5 ug/kg, 1 ug/kg, 0.5 ug/kg, 0.3 ug/kg, 0.25 ug/kg, 0.15 ug/kg or 0.1 ug/kg or a combination thereof;
(iii) the target dose is 6 ug/kg and the one or more priming doses are 3 ug/kg, 1.5 ug/kg, 1 ug/kg, 0.5 ug/kg, 0.3 ug/kg, 0.25 ug/kg, 0.15 ug/kg or 0.1 ug/kg or a combination thereof;
(iv) the target dose is 12 ug/kg and the one or more priming doses are 6 ug/kg, 3 ug/kg, 1.5 ug/kg, 1 ug/kg, 0.5 ug/kg, 0.3 ug/kg, 0.25 ug/kg, 0.15 ug/kg or 0.1 ug/kg or a combination thereof;
(v) the target dose is 18 ug/kg and the one or more priming doses are 9 ug/kg, 4.5 ug/kg, 6 ug/kg, 3 ug/kg, 1.5 ug/kg, 1 ug/kg, 0.5 ug/kg, 0.3 ug/kg, 0.25 ug/kg, 0.15 ug/kg or 0.1 ug or a combination thereof; or
(vi) the target dose is 24 ug/kg and the one or more priming doses are 12 ug/kg, 9 ug/kg, 4.5 ug/kg, 6 ug/kg, 3 ug/kg, 1.5 ug/kg, 1 ug/kg, 0.5 ug/kg, 0.3 ug/kg, 0.25 ug/kg, 0.15 ug/kg or 0.1 ug or a combination thereof; or
(vii) the target dose is 1.5 ug/kg, 3 ug/kg, 6 ug/kg, 12 ug/kg, 20 ug/kg, or 30 ug/kg and the one or more priming doses are 0.25 ug/kg, 1.5 ug/kg, 3 ug/kg, 6 ug/kg, 12 ug/kg, and 20 ug/kg (the skilled artisan will appreciate that the if the target dose is 1.5 ug/kg, the priming dose will be selected from a value less than 1.5 ug/kg).

Particularly preferred administration regimens include (a) providing the IL-2 receptor agonist on a 21 day cycle wherein a priming dose is administered on day 1 and on day 8 of the first 21 day cycle and the target dose is providing on days 1 and 8 of two or more subsequent 21 day cycles or (b) providing the IL-2 receptor agonist on a 21 day cycle wherein a priming dose is administered on day 1 and day 8 of the first 21 day cycle and the target dose level is provided on day 1 of two or more subsequent 21 day cycles. Administration can be, for example, as an intravenous infusion.

### EXAMPLES

### Example 1: Step Dosing in CT-26 Murine Tumor Model

The Second Dose of the Test Article is Better Tolerated than the First Dose

BALB/c mice were implanted with CT-26 cells. When tumors were palpable (100 mm³), mice were grouped and treatment with the IL-2 receptor agonist (PEGylated E62C NEO 2-15) began. Mice were administered with 150, 300, 600, or 1200 ug/kg of the test article on Study Day 10. A second dose was administered to surviving mice on Study Day 17. Body weights and tumor volumes were monitored twice weekly on all surviving animals. Mice were sacrificed when tumor volume reached 3000 mm³ or body weight loss of greater than 15% was observed. The point of maximum toxicity was observed approximately 4 days following dose administration as evidenced by body weight loss reaching a maximum at this time.

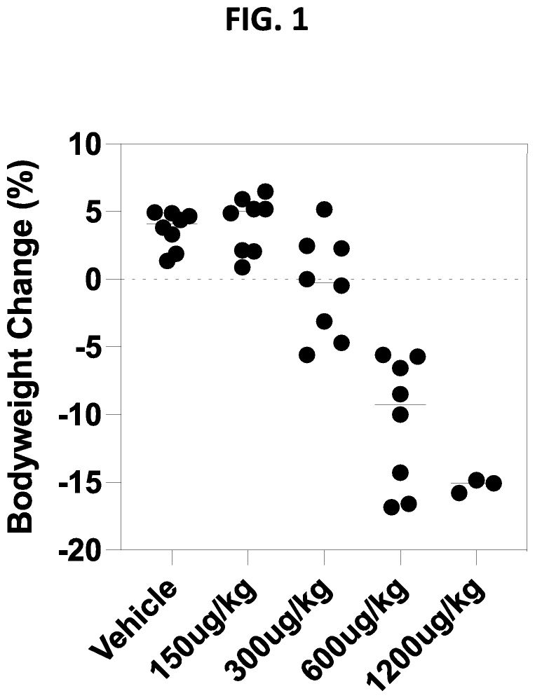

All mice receiving 150, 300, or 600 ug/kg of the test article survived both doses, while all mice receiving 1200 ug/kg were either found dead or moribund on Day 14. The maximum tolerated dose was established at 600 ug/kg. For surviving animals, the first dose of the test article resulted in a dose-dependent change in body weight on Day 14 vs. Day 10 of +3.66% (vehicle), +4.10% (150 ug/kg), −0.49% (300 ug/kg), or −10.51% (600 ug/kg) (See FIG. 1). In contrast, in the first four days after the second dose (i.e., between days 17 and 21), mice experienced an average change in body weight of +4.80% (vehicle), +2.66% (150 ug/kg), +1.77% (300 ug/kg), or +1.26% (600 ug/kg) (data not shown). These results demonstrate that the second dose of the test article is better tolerated than the first.

The Maximum Tolerated Second Dose is Higher than the Maximum Tolerated First Dose

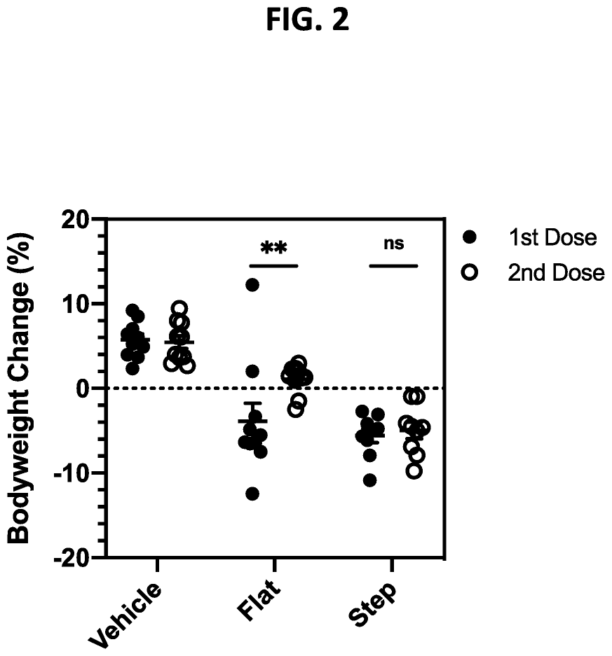

BALB/c mice bearing CT-26 tumors were grouped (on Study Day 9) into two cohorts: one receiving 500 ug/kg on Day 9 and 500 ug/kg on Day 16 (baseline or flat dosing), and one receiving 500 ug/kg on Day 9 and 1000 ug/kg on Day 16 (step-up or step dosing). After the first dose, mice experienced a body weight change of −3.87% (baseline) and −5.58% (step) respectively on Day 13 vs Day 9 (FIG. 2). After the second dose, mice receiving 500 ug/kg (baseline) experienced a body weight change of +1.10% on Day 20 vs Day 16, while mice receiving 1000 ug/kg (step-up) experienced an average body weight change of −5.18%. All mice receiving both 500 ug/kg and 1000 ug/kg survived the second dose (See FIG. 2). This result illustrates that a second dose of 1000 ug/kg was approximately as well tolerated as a first dose of 500 ug/kg, supporting the observation that the maximum tolerated second dose is higher than the maximum tolerated first dose. The reason for greater maximum tolerated second dose vs. first dose may be due to differences in cytokine release, cytokine receptor expression, and/or the status of immune cell populations at the time of dosing as a result of the administration of the first dose.

Therapeutic Benefit from Step-Dosing

To determine whether an increase in the second dose leads to a therapeutic benefit, tumor size was tracked over time in both the baseline and step-up cohorts. One mouse in the step-up cohort was found dead on Study Day 13 (prior to the second dose) and was excluded from analysis. 4 out of 10 (40%) mice in the baseline cohort showed lack of tumor control by Study Day 55 (characterized by tumor volume reaching tumor volume limits) while 2 out of 9 (22%) mice in the step-up cohort showed lack of tumor control over the same time period. In contrast, 6 out of 10 (60%) mice in the baseline cohort showed tumor control (characterized by tumor size reaching <1000 mm3), while 7 out of 9 mice (77.7%) in the step-up cohort had controlled tumors (data not shown). All mice in the control arm died due to tumor growth. The mean tumor volumes were 233+/−61 mm³ for the baseline cohort and 161+/−74 mm³ for the step up cohort. In larger cohorts and/or with longer treatment duration, it is believed that a difference in tumor control between groups will become more prominent.

### Example 2: Preparation of Test Article

Neo-2/15 stocks with a single E62C mutation (SEQ ID NO:20) were dialyzed into phosphate buffer, pH7.0 and adjusted to 1.0-2.0 mg/ml. TCEP was added at a molar ratio of 10:1 to protein and incubated for 10 minutes at RT to reduce disulfides. Maleimide-modified PEG40k (PEG40k-MA) or PEG30k (PEG30k-MA) powder was added directly to the reduced protein solution at a molar ratio of 10:1 PEG:cysteine and incubated for 2 hours with stirring. Aliquots for SDS-PAGE were taken directly from the reaction mixture. These data demonstrate the rapid, spontaneous, and near-quantitative formation of covalent linkages between PEG40k-MA or PEG30k-MA and Neo-2/15 cysteine mutants in the expected stoichiometry.

### Example 3: Exemplary Dose Escalation Scheme

In this example, there are two schedules for dosing. Dosing is on day 1 of a 21 day cycle or on days 1 and 8 of a 21 day cycle. Patients in Schedule A will be administered the priming dose on days 1 and 8 of the first 21 day cycle and resume dosing on day 1 of every 21 day cycle for the subsequent cycles. Patients in Schedule B will continue dosing on days 1 and 8 of a 21 day cycle. Administration is as an intravenous infusion. The drug is PEGylated E62C NEO 2-15 polypeptide. The cycle 1 dose used for step-dosing for this example will be calculated as one half of the highest tolerated initial dose (HTID), i.e. the highest dose at which <33% of patients experience DLTs. Subsequent cycles will increase by one dose level per cycle until the cohort target dose (CTD) is reached. Table 3 indicates exemplary doses (in μg/kg) that would be given during step-dosing assuming 0.5 μg/kg is the HTID.

TABLE 3
Doses for each cohort target dose to be given during
an exemplary step-dosing (in μg/kg)

| Cohort | First Cycle | | Subsequent cycles | | | | | |
|---|---|---|---|---|---|---|---|---|
| dose | (μg/kg) | | (μg/kg)² | | | | | |
| target | D1 | D8 | C2 | C3 | C4 | C5 | C6 | C7 |
| 1.5 μg/kg | 0.25 | 0.25 | 1.5 | 1.5 | 1.5 | 1.5 | 1.5 | 1.5 |
| 3 μg/kg | 0.25 | 0.25 | 1.5 | 3 | 3 | 3 | 3 | 3 |
| 6 μg/kg | 0.25 | 0.25 | 1.5 | 3 | 6 | 6 | 6 | 6 |
| 12 μg/kg | 0.25 | 0.25 | 1.5 | 3 | 6 | 12 | 12 | 12 |
| 20 μg/kg | 0.25 | 0.25 | 1.5 | 3 | 6 | 12 | 20 | 20 |
| 30 μg/kg | 0.25 | 0.25 | 1.5 | 3 | 6 | 12 | 20 | 30 |

²Schedule A patients will receive the indicated dose on DI of each 21 day cycle, while Schedule B patients will receive the indicated dose on D1 and D8 of each 21 day cycle.

What is claimed is:

1. A method for (i) modulating an immune response or (ii) treating cancer in a subject in need thereof with an IL-2 receptor agonist comprising (a) administering to the subject one or more priming doses of the IL-2 receptor agonist in order to enable escalation to a target dose level that would have an unacceptable tolerability profile if administered to the subject as a first dose, and (b) administering to the subject the IL-2 receptor agonist at the target dose level.

2. The method of claim 1 wherein the IL-2 receptor agonist is long-acting.

3. A method for (i) modulating an immune response or (ii) treating cancer in a subject in need thereof with a long-acting IL-2 receptor agonist comprising (a) administering to the subject one or more priming doses of the IL-2 receptor agonist in order to enable escalation to a target dose level that would have an unacceptable tolerability profile if administered to the subject as a first dose, and (b) administering to the subject the IL-2 receptor agonist at the target dose level.

4. A method for (i) modulating an immune response or (ii) treating cancer in a subject in need thereof with a dosing regimen of an IL-2 receptor agonist comprising administering to the subject one or more priming doses of the IL-2 receptor agonist at one or more priming dose levels following by administration of the IL-2 receptor agonist at a target dose level wherein the target dose level is greater than the priming dose levels.

5. The method of claim 4 wherein the IL-2 receptor agonist is long-acting.

6. A method for (i) modulating an immune response or (ii) treating cancer in a subject in need thereof with a dosing regimen of a long-acting IL-2 receptor agonist comprising administering to the subject one or more priming doses of the IL-2 receptor agonist at one or more priming dose levels following by administration of the IL-2 receptor agonist at a target dose level wherein the target dose level is greater than the priming dose levels.

7. A method for (i) modulating an immune response or (ii) treating cancer in a subject in need thereof with a long-acting IL-2 receptor agonist comprising (i) selecting a target dose and a priming dose for administration to the subject; wherein the target dose is associated with an unacceptable tolerability profile if administered to the subject as a first dose but has a more favorable tolerability profile if administered after a priming dose (ii) administering one or more of the priming doses of the IL-2 receptor agonist to the subject in order to enable escalation to the target dose level, and administering to the subject the IL-2 receptor agonist at the target dose level.

8. The method of any one of the preceding claims wherein a first administration of the IL-2 receptor agonist at the target dose level is at least 5, 6 or 7 days following administration of the first priming dose.

9. The method of any one of the preceding claims wherein a first administration of the IL-2 receptor agonist at the target dose level is no more than 21 days following administration of a priming dose.

10. The method of claim 9 wherein a first administration of the IL-2 receptor agonist at the target dose level is no more than 21 days following administration of a first priming dose.

11. The method of any one of claims 1 to 7 wherein a first administration of the IL-2 receptor agonist at the target dose level is at any one of days 7 to 21, or 7 to 63 following administration of a priming dose.

12. The method of claim 11 wherein a first administration of the IL-2 receptor agonist at the target dose level is at any one of days 7 to 21, or 7 to 63 following administration of a first priming dose.

13. The method of any of the preceding claims wherein one to six priming doses are administered to the subject.

14. The method of any of the preceding claims wherein one to three priming doses are administered to the subject.

15. The method of any of the preceding claims wherein two priming doses are administered to the subject.

16. The method of claim 15 wherein two priming doses are administered to the subject and the priming doses are administered on day 1 and day 8 of a first 21 day treatment cycle.

17. The method of claim 16 wherein the target dose is administered to the subject at days 1 and 8 of two or more subsequent 21 day treatment cycles or at day 1 of two or more subsequent 21 day treatment cycles.

18. The method of any one of the preceding claims wherein more than one priming dose is administered to the subject and the priming doses of the IL-2 receptor agonist are at escalating dose levels.

19. The method of any one of claims 1 to 17 wherein more than one priming dose is administered to the subject and the priming doses of the IL-2 receptor agonist are at the same dose levels.

20. The method of any one of claims 1 to 17 wherein the initial priming dose is at a dose level that is greater than the subsequent priming doses.

21. The method of any one of the preceding claims wherein the priming doses are provided over a period of 1-10 days.

22. The method of any of claims 1 to 14 wherein one priming dose is administered to the subject.

23. The method of any one of the preceding claims wherein the target dose level is at least 25%, at least 50%, or at least 75% greater than the initial priming dose level.

24. The method of any one of claims 1 to 22 wherein the target dose level is double the initial priming dose level.

25. The method of any one of claims 1 to 22 wherein the target dose level is no more than double the initial priming dose level.

26. The method of any one of claims 1 to 22 wherein the target dose level is at least triple the initial priming dose level.

27. The method of any one of claims 1 to 22 wherein the target dose level is at least four to six times the initial priming dose level.

28. The method of any one of claims 1 to 22 wherein the target dose level is at least ten times the initial priming dose level.

29. The method of any one of claims 1 to 22 wherein the target dose level is at least 25%, at least 50%, or at least 75% greater than each of the priming dose levels.

30. The method of any one of claims 1 to 22 wherein the target dose level is at least double each of the priming doses levels.

31. The method of any one of the preceding claims wherein following the first administration of the IL-2 receptor agonist at the target dose level, subsequent doses are administered at the target dose level.

32. The method of any one of the preceding claims wherein following the first administration of the IL-2 receptor agonist at the target dose level, two to eight subsequent doses are administered at the target dose level at intervals of between 7 to 21 days.

33. The method of any one of the preceding claims wherein the one or more priming doses are from 0.01 ug/kg to 1 mg/kg.

34. The method of any one of the preceding claims wherein the target dose level is from 0.1 ug/kg to 2 mg/kg.

35. The method of any one of claims 1 to 32 wherein the one or more priming doses are from 0.1 ug/kg to 12 ug/kg.

36. The method of any one of claims 1 to 32 wherein the one or more priming doses are from 0.1 ug/kg to 10 ug/kg.

37. The method of any one of claims 1 to 32 wherein the one or more priming doses are from 0.1 ug/kg to 5 ug/kg.

38. The method of any one of claims 1 to 32 wherein the one or more priming doses are from 0.1 ug/kg to 2 ug/kg.

39. The method of any one of claims 1 to 32 wherein the one or more priming doses are from 0.5 ug/kg to 20 ug/kg.

40. The method of any one of claims 1 to 32 wherein the one or more priming doses are from 0.5 ug/kg to 10 ug/kg.

41. The method of any one of claims 1 to 32 wherein the one or more priming doses are from 0.5 ug/kg to 3 ug/kg.

42. The method of any one of claim 1 to 32 or 35-38 wherein the target dose level is from 0.2 ug/kg to 1 mg/kg.

43. The method of any one of claim 1 to 32 or 35-38 wherein the target dose level is from 0.2 ug/kg to 50 ug/kg.

44. The method of any one of claim 1 to 32 or 35-38 wherein the target dose level is from 0.2 ug/kg to 20 ug/kg.

45. The method of any one of claim 1 to 32 or 35-38 wherein the target dose level is from 0.2 ug/kg to 10 ug/kg.

46. The method of any one of claim 1 to 32 or 35-38 wherein the target dose level is from 0.2 ug/kg to 8 ug/kg.

47. The method of any one of claim 1 to 32 or 35-41 wherein the target dose level is from 1 ug/kg to 50 ug/kg.

48. The method of any one of claim 1 to 32 or 35-41 wherein the target dose level is from 1.5 ug/kg to 30 ug/kg.

49. The method of any one of claim 1 to 32 or 35-41 wherein the target dose level is from 2 ug/kg to 35 ug/kg.

50. The method of any one of claim 1 to 32 or 35-41 wherein the target dose level is from 4 ug/kg to 20 ug/kg.

51. The method of any one of claims 1 to 32 wherein the one more priming doses are from 0.1 ug/kg to 10 ug/kg, from 0.1 ug/kg to 5 ug/kg, or from 0.1 ug/kg to 2 ug/kg and the target dose is from 0.2 ug/kg to 20 ug/kg; the one or more priming doses are from 0.1 ug/kg to 5 ug/kg or from 0.1 ug/kg to 2 ug/kg and the target dose is from 0.2 ug/kg to 20 ug/kg, from 0.2 ug/kg to 10 ug/kg, or from 0.2 ug/kg to 8 ug/kg; the one or more priming doses are from 0.5 ug/kg to 10 ug/kg, or from 0.5 ug/kg to 3 ug/kg and the target dose is from 2 ug/kg to 35 ug/kg; the one or more priming doses are from 0.5 ug/kg to 10 ug/kg or from 0.5 ug/kg to 3 ug/kg and the target dose is from 2 ug/kg to 35 ug/kg; the one or more priming doses are from 0.5 ug/kg to 10 ug/kg or from 0.5 ug/kg to 3 ug/kg and the target dose is from 4 ug/kg to 20 ug/kg; the one or more priming doses are from 0.25 ug/kg to 20 ug/kg and the target dose is from 1.5 ug/kg to 30 ug/kg; or the one or more priming doses are from 0.25 ug/kg to 4.5 ug/kg and the target dose is from 1.5 ug/kg to 10 ug/kg; provided that the target dose is greater than the priming dose.

52. The method of any one of claims 1 to 32 wherein the target dose is 1 ug/kg and the one or more priming doses are 0.1 ug/kg, 0.15 ug/kg, 0.25 ug/kg, 0.3 ug/kg, or 0.5 ug/kg or a combination thereof; the target dose is 3 ug/kg and the one or more priming doses are 1.5 ug/kg, 1 ug/kg, 0.5 ug/kg, 0.3 ug/kg, 0.25 ug/kg, 0.15 ug/kg or 0.1 ug/kg or a combination thereof; the target dose is 6 ug/kg and the one or more priming doses are 3 ug/kg, 1.5 ug/kg, 1 ug/kg, 0.5 ug/kg, 0.3 ug/kg, 0.25 ug/kg, 0.15 ug/kg or 0.1 ug/kg or a combination thereof; the target dose is 12 ug/kg and the one or more priming doses are 6 ug/kg, 3 ug/kg, 1.5 ug/kg, 1 ug/kg, 0.5 ug/kg, 0.3 ug/kg, 0.25 ug/kg, 0.15 ug/kg or 0.1 ug/kg or a combination thereof; the target dose is 18 ug/kg and the one or more priming doses are 9 ug/kg, 4.5 ug/kg, 6 ug/kg, 3 ug/kg, 1.5 ug/kg, 1 ug/kg, 0.5 ug/kg, 0.3 ug/kg, 0.25 ug/kg, 0.15 ug/kg or 0.1 ug or a combination thereof; the target dose is 24 ug/kg and the one or more priming doses are 12 ug/kg, 9 ug/kg, 4.5 ug/kg, 6 ug/kg, 3 ug/kg, 1.5 ug/kg, 1 ug/kg, 0.5 ug/kg, 0.3 ug/kg, 0.25 ug/kg, 0.15 ug/kg or 0.1 ug or a combination thereof; or the target dose is 1.5 ug/kg, 3 ug/kg, 6 ug/kg, 12 ug/kg, 20 ug/kg, or 30 ug/kg and the one or more priming doses are 0.25 ug/kg, 1.5 ug/kg, 3 ug/kg, 6 ug/kg, 12 ug/kg, or 20 ug/kg provided that the target dose is greater than the priming dose.

53. The method of any one of the preceding claims wherein the IL-2 receptor agonist is a recombinantly engineered IL-2 receptor agonist.

54. The method of any one of the preceding claims wherein the IL-2 receptor agonist has a plasma or serum half-life of 6 hours or greater.

55. The method of any one of the preceding claims wherein the IL-2 receptor agonist has a plasma or serum half-life of 10 hours or greater.

56. The method of any one of the preceding claims wherein the IL-2 receptor agonist is an IL-2 polypeptide.

57. The method of claim 56 wherein the IL-2 polypeptide has at least 85% sequence identity to the amino acid sequence of human IL-2.

58. The method of any one of the preceding claims wherein the IL-2 receptor agonist is an IL-2 polypeptide mimetic.

59. The method of claim 58 wherein the IL-2 receptor agonist mimetic is non-naturally occurring polypeptide comprising domains X1, X2, X3, and X4, wherein:
(a) X1 is a peptide comprising an amino acid sequence at least 25%, 27%, 30%, 35%, 40%, 45%, 50%, 55%, 60%, 65%, 70%, 75%, 80%, 85%, 90%, 95%, 98%, or 100% identical to EHALYDAL (SEQ ID NO:1);
(b) X2 is a helical-peptide of at least 8 amino acids in length;
(c) X3 is a peptide comprising an amino acid sequence at least 25%, 27%, 30%, 35%, 40%, 45%, 50%, 55%, 60%, 65%, 70%, 75%, 80%, 85%, 90%, 95%, 98%, or 100% identical to YAFNFELI (SEQ ID NO:2);
(d) X4 is a peptide comprising an amino acid sequence at least 25%, 27%, 30%, 35%, 40%, 45%, 50%, 55%, 60%, 65%, 70%, 75%, 80%, 85%, 90%, 95%, 98%, or 100% identical to ITILQSWIF (SEQ ID NO:3);
wherein X1, X2, X3, and X4 may be in any order in the polypeptide;
wherein amino acid linkers may be present between any of the domains; and
wherein the polypeptide binds to IL-2 receptor β [symbol] _{c} heterodimer (IL-WYO.

60. The method of claim 58 wherein the IL-2 receptor agonist mimetic is non-naturally occurring polypeptide comprising domains X1, X2, X3, and X4, wherein:
(a) X1 is a peptide comprising an amino acid sequence at least 85% identical to EHALYDAL (SEQ ID NO:1);
(b) X2 is a helical-peptide of at least 8 amino acids in length;
(c) X3 is a peptide comprising an amino acid sequence at least 85% identical to YAFNFELI (SEQ ID NO:2);
(d) X4 is a peptide comprising an amino acid sequence at least 85% identical to ITILQSWIF (SEQ ID NO:3);
wherein X1, X2, X3, and X4 may be in any order in the polypeptide;
wherein amino acid linkers may be present between any of the domains; and
wherein the polypeptide binds to IL-2 receptor β [symbol] _{c} heterodimer (IL-2Rβ [symbol] _{c}).

61. The method of claim 58, 59, or 60 wherein the IL-2 receptor agonist mimetic is a non-naturally occurring polypeptide comprising domains X1, X2, X3, and X4, wherein:
X1 is a peptide comprising an amino acid sequence at least 25%, 27%, 30%, 35%, 40%, 45%, 50%, 55%, 60%, 65%, 70%, 75%, 80%, 85%, 90%, 95%, 98%, or 100% identical along its length to the peptide PKKKIQLHAEHALYDALMILNI (SEQ ID NO: 4);
X2 is a helical-peptide of at least 8 amino acids in length;
X3 is a peptide comprising an amino acid sequence at least 25%, 27%, 30%, 35%, 40%, 45%, 50%, 55%, 60%, 65%, 70%, 75%, 80%, 85%, 90%, 95%, 98%, or 100% identical along its length the peptide LEDYAFNFELILEEIARLFESG (SEQ ID NO:5); and
X4 is a peptide comprising an amino acid sequence at least 25%, 27%, 30%, 35%, 40%, 45%, 50%, 55%, 60%, 65%, 70%, 75%, 80%, 85%, 90%, 95%, 98%, or 100% identical along its length to the peptide EDEQEEMANAIITILQSWIFS (SEQ ID NO:6) wherein X1, X2, X3, and X4 may be in any order in the polypeptide; wherein amino acid linkers may be present between any of the domains; and wherein the polypeptide binds to IL-2 receptor β [symbol] _{c} heterodimer (IL-2Rβ [symbol] _{c}).

62. The method of claim 61 wherein the IL-2 receptor agonist mimetic is a non-naturally occurring polypeptide comprising domains X1, X2, X3, and X4, wherein:
X1 is a peptide comprising an amino acid sequence at least 70% identical along its length to the peptide PKKKIQLHAEHALYDALMILNI (SEQ ID NO: 4);
X3 is a peptide comprising an amino acid sequence at least 70% identical along its length the peptide LEDYAFNFELILEEIARLFESG (SEQ ID NO:5); and
X4 is a peptide comprising an amino acid sequence at least 70% identical along its length to the peptide EDEQEEMANAIITILQSWIFS (SEQ ID NO:6).

63. The method of claim 61 wherein the IL-2 receptor agonist mimetic is a non-naturally occurring polypeptide comprising domains X1, X2, X3, and X4, wherein:
X1 is a peptide comprising an amino acid sequence at least 80% identical along its length to the peptide PKKKIQLHAEHALYDALMILNI (SEQ ID NO: 4);
X3 is a peptide comprising an amino acid sequence at least 80% identical along its length the peptide LEDYAFNFELILEEIARLFESG (SEQ ID NO:5); and
X4 is a peptide comprising an amino acid sequence at least 80% identical along its length to the peptide EDEQEEMANAIITILQSWIFS (SEQ ID NO:6).

64. The method of claim 61 wherein the IL-2 receptor agonist mimetic is a non-naturally occurring polypeptide comprising domains X1, X2, X3, and X4, wherein:
X1 is a peptide comprising an amino acid sequence at least 90% identical along its length to the peptide PKKKIQLHAEHALYDALMILNI (SEQ ID NO: 4);
X3 is a peptide comprising an amino acid sequence at least 90% identical along its length the peptide LEDYAFNFELILEEIARLFESG (SEQ ID NO:5); and
X4 is a peptide comprising an amino acid sequence at least 90% identical along its length to the peptide EDEQEEMANAIITILQSWIFS (SEQ ID NO:6).

65. The method of any one of claims 59-64, wherein X2 is a peptide comprising an amino acid sequence at least 25%, 27%, 30%, 35%, 40%, 45%, 50%, 55%, 60%, 65%, 70%, 75%, 80%, 85%, 90%, 95%, 98%, or 100% identical along its length to KDEAEKAKRMKEWMKRIKT (SEQ ID NO:7).

66. The method of any one of claims 58-65 wherein a cysteine residue is present within the polypeptide and is attached to a stabilization moiety.

67. The method of any one of claims 58-65 wherein an amino acid residue of X1, X2, X3 or X4 is mutated to a cysteine residue for attachment of a stabilization moiety.

68. The method of claim 67 wherein the cysteine is present in the X2 domain.

69. The method of claim 68 wherein the cysteine is present at positions 1 (K58C), 2 (D59C), 5 (E62C), 9 (R66C), 12 (E69C) or 16 (R73C) of the X2 domain relative to SEQ ID NO:7.

70. The method of any one of claims 66 to 69 wherein the stabilization moiety is a PEG-containing moiety.

71. The method of claim 70 wherein the stabilization moiety is a maleimide modified PEG-moiety.

72. The method of any one of claims 58 to 71 wherein the order of X1, X2, X3, and X4 is X1-X3-X2-X4.

73. The method of any one of claims 58 to 72, wherein the IL-2 receptor agonist mimetic comprises a polypeptide at least 25%, 27%, 30%, 35%, 40%, 45%, 50%, 55%, 60%, 65%, 70%, 75%, 80%, 85%, 90%, 95%, 98%, or 100% identical to the amino acid sequence set forth in SEQ ID NO:8, or 9.

74. The method of any one of claims 58 to 72, wherein the IL-2 receptor agonist mimetic comprises a polypeptide at least 25%, 27%, 30%, 35%, 40%, 45%, 50%, 55%, 60%, 65%, 70%, 75%, 80%, 85%, 90%, 95%, 98%, or 100% identical to the full length of the amino acid sequence of SEQ ID NO:8 or SEQ ID NO:9, but for one, two, or more of the following residues are mutated to cysteine:
amino acid residue 1, 2, 5, 9, 12, or 16 in the X2 domain wherein numbering is according to SEQ ID NO:7,
amino acid residues 17 or 20 in the X3 domain wherein numbering is according to SEQ ID NO:5,
amino acid residues 3 or 6 in the X4 domain wherein numbering is according to SEQ ID NO: 6.

75. The method of any one of claims 58 to 77 wherein the IL-2 receptor agonist mimetic comprises a polypeptide at least 80% identical along its length to an amino acid sequence selected from any one of SEQ ID NOs:10-33.

76. The method of claim 75 wherein the IL-2 receptor agonist mimetic comprises a polypeptide identical along its length to an amino acid sequence selected from any one of SEQ ID NOs:10-33.

77. The method of any one of claims 58 to 76, wherein the polypeptide is linked to a stabilization compound, including but not limited to a polyethylene glycol (“PEG”) containing moiety.

78. The method of claim 77, wherein the stabilization compound is linked at a cysteine residue in the polypeptide.

79. The method of claim 78, wherein the stabilization compound is a PEG-containing moiety.

80. The method of claim 79 wherein the amino acid sequence is SEQ ID NO:20 and the cysteine at position 62 is present and is linked to the stabilization compound, the amino acid sequence is SEQ ID NO:30 and the cysteine at position 82 is present and is linked to the stabilization compound, amino acid sequence is SEQ ID NO:24 and the cysteine at position 69 is present and is linked to the stabilization compound, or amino acid sequence is SEQ ID NO:26 and the cysteine at position 73 is present and is linked to the stabilization compound.

81. The method of any one of claims 77 to 80 wherein the stabilization compound is linked to the cysteine reside via a maleimide group.

82. The method of any one of claims 1 to 65 wherein the IL-2 receptor agonist is conjugated to a water-soluble polymer.

83. The method of claim 82 wherein the IL-2 receptor agonist is PEGylated.

84. The method of any one of the preceding claims wherein the IL-2 receptor agonist is not targeted.

85. The method of any one of the preceding claims wherein the IL-2 receptor agonist is not a fusion protein.

86. The method of any one of claims 1 to 83 wherein the IL-2 receptor agonist is conjugated to a targeting domain.

87. The method of claim 86 wherein the targeting domain is an antibody.

88. The method of any one the preceding claims wherein the method is for modulating an immune response.

89. The method of claim 88 wherein the immune response is an anti-cancer immune response.

90. The method of any one of the previous claims wherein the method is for the treatment of cancer.

91. The method of claim 89 or 90 wherein the cancer is a metastatic cancer.

92. The method of claim 89 or 90 wherein the cancer is renal cell carcinoma.

93. The method of claim 89 or 90 wherein the cancer is melanoma.

94. The method of claim 89 or 90 wherein the cancer is colorectal cancer, breast cancer, lung cancer, a sarcoma, head and neck cancer, liver cancer or bladder cancer.

95. The method of claim 89 or 90 wherein the cancer is a solid tumor.

96. The method of any one of the preceding claims wherein the doses are administered by IV or subcutaneous injection.

97. The method of any one of the preceding claims wherein the IL-2 receptor agonist is a β [symbol] _{c} selective IL-2 receptor agonist.

98. The method of any one of the preceding claims wherein the IL-2 receptor agonist is PEGylated and wherein the number of repeating PEG units in the PEG-containing moiety is about 800-1000.

99. The method of any one of the preceding claims wherein the IL-2 receptor agonist is PEGylated and wherein the average number of repeating PEG units in the PEG-containing moiety is about 850-950.

100. The method of any one of claim 98 or 99 wherein the PEG is a linear PEG.

101. The method of any one of claim 98 or 99 wherein the PEG is a branched PEG.
